(19) United States
(12) Patent Application Publication
(10) Pub. No.: US 2012/0167879 A1
(43) Pub. Date: Jul. 5, 2012

(54) POSITIVE AIRWAY PRESSURE THERAPY APPARATUS AND METHODS

(76) Inventors: Bruce R. Bowman, Eden Prairie, MN (US); Eric Allan Becker, Brooklyn Park, MN (US)

(21) Appl. No.: 13/339,870

(22) Filed: Dec. 29, 2011

Related U.S. Application Data
(60) Provisional application No. 61/430,463, filed on Jan. 6, 2011.
(60) Provisional application No. 61/429,181, filed on Jan. 3, 2011.

(51) Int. Cl. A61M 16/06; A61M 16/00

(52) U.S. Cl. 128/201.22; 128/205.25; 128/205.12

(57) ABSTRACT

Embodiments of the invention are directed to positive airway pressure therapy apparatus and methods for using same. In some embodiments, the apparatus includes a body- (e.g., head-) mounted housing enclosing a blower, wherein the housing includes air inlets on multiple sides so that the blower may draw adequate air even when inlets on one side are obstructed. In another embodiment, the apparatus includes a mask or mask system securable to an airway of the user. The mask system may include a mask shell and two or more adapters configured to permit the attachment of two or more different mask seals to the same mask shell. Other embodiments of the invention describe strapping systems configured to removably attach the housing and the mask to the body. The apparatus may further include a drying mode that activates upon removal of the apparatus from the user.

### RELATED APPLICATION(S)

This application claims the benefit of U.S. Provisional Application No. 61/430,463, filed Jan. 6, 2011, and U.S. Provisional Application No. 61/429,181, filed Jan. 3, 2011, both of which are incorporated herein by reference in their respective entireties. All other documents identified herein are incorporated by reference herein in their respective entireties as if each were individually incorporated.

Embodiments of the present invention relate to positive airway pressure therapy apparatus, and to methods for providing positive airway pressure respiratory therapy to a user.

### BACKGROUND

Positive airway pressure therapies are frequently used in the treatment of, among other ailments, obstructive sleep apnea, asthma, bronchitis, chronic obstructive pulmonary disease (COPD), snoring, and congestive heart failure. These therapies typically pressurize the airway of a user to a pressure in the range of 4-30 centimeters (cm) of water (e.g., often about 4-20 cm water) or more. Depending upon the particular therapy, a variable or a constant pressure therapy may be administered to the user to reduce or eliminate airway occlusions (or to otherwise treat acute or chronic respiratory failure) that necessitated the use of the therapy.

Typically, therapeutic devices used to provide positive airway pressure therapy include at least a blower unit, an elongated hose, and a mask. The blower unit is frequently a relatively large component which rests on a bedside table or floor adjacent the bed. The blower typically includes a fan or turbine, a motor, and associated controls. Accordingly, the blower may be heavy and, while operating, potentially noisy. The elongated hose is typically configured to span a distance between the user's head and the location at which the blower unit resides (e.g., night-stand or floor). Accordingly, the first end of the elongated tube is generally connected to an outlet of the blower and the second end is typically connected to the mask. The mask is generally configured to be secured relative to a user's head and to deliver or communicate the pressurized air from the blower to the airway of the user during sleep.

In many instances, the noise from the blower, which may travel through the hose, may disrupt sleep of the user or others who are in the same bed or in the same room as the blower. In some instances, the noise can even disrupt light sleepers in other rooms.

Pressure is typically monitored by the positive airway pressure device near the outlet of the blower and controlled by a controller of the device in a way that maintains a desired pressure during breathing. The face (e.g., the head) of the user is tethered by the hose to the remotely positioned blower. The hoses associated with typical positive airway pressure therapy devices are about six feet long. The longer the hose and the narrower the inside diameter of the hose, the larger the potential fluctuation in pressure along the length of the hose can be during breathing. That is, for a long and/or small diameter hose, the blower may have difficulty adequately maintaining the desired regulation of pressure while breathing. Often, during inspiration, the positive airway pressure therapy device may actually deliver a lower pressure at the user's mask than intended. Likewise, during exhalation, the positive airway pressure therapy device may actually deliver a higher pressure at the user's mask than intended.

Another issue with known blower devices is that the placement of the blower may be limited by the hose and by the connection point (outlet) of the blower, as well as by the air inlet location on the mask. Moreover, the hose length can pose a nuisance for storage. Still further, due to placement of the blower relative to the user, the fixed length of the hose frequently proves to be either too long or too short, generating slack or tension, respectively, between the remotely positioned blower and the user's head. As a result, portions of the hose may become entangled in the bedding and inadvertently move the blower and/or displace the mask. Either condition may limit a user's ability to freely change head and body positions during sleep, which may prove disruptive. Moreover, even if one is able to change body positions, resulting tension or compression in the tube can torque the mask, ultimately producing mask leaks. In some instances, these leaks may adversely affect the pressure in the mask (and thus the therapeutic efficacy of the system), and further result in user discomfort. Yet another problem with excessive lengths of hose is that the hose may become inadvertently entangled with the user (or the user's partner) during sleep.

Still further, the bulk of typical positive pressure therapeutic devices, as well as the length of the accompanying hose, may result in at least the perception that they are cumbersome to use and difficult to transport. Moreover, problems created by divergent placement locations for the blower relative to the bed may be presented in different sleeping accommodations, resulting, once again, in a hose that may be too long or too short.

### SUMMARY

The present invention provides apparatus and methods for the administration of positive airway pressure therapy to a mask that is configured for fluid communication with an airway (air passageway) of a user. Embodiments of the present invention may provide a therapy apparatus wherein at least the blower is body- (e.g., head-) mounted. Such systems may, as compared to more conventional designs, provide a shortened hose and/or provide monitoring of pressure directly at the mask instead of at the outlet of the blower. Such embodiments may reduce or eliminate undesirable pressure fluctuations that are common with conventional therapy apparatus. Apparatus in accordance with embodiments of the present invention may also include strapping systems for holding or removably attaching a main portion (e.g., a blower) of a positive airway pressure therapy apparatus to the head of the user. The positive airway pressure therapy apparatus may also include noise abatement mechanisms, as well as other features that are advantageous to bodily mounting of some or all of the apparatus. Other embodiments are directed to other features and aspects as shown and described herein.

In one embodiment, a positive airway pressure therapy apparatus is provided that may include: a device comprising a housing enclosing a blower, the housing configured to attach to a user's body, wherein the housing comprises at least two distinct side surfaces, and wherein each side surface defines an air inlet fluidly coupled to the blower; a mask configured to communicate pressurized air generated by the blower to an airway of the user; and a delivery tube extending from the housing to the mask, the delivery tube defining an air delivery passage configured to communicate the pressurized air from an outlet of the housing to an inlet of the mask. One or more aspects may be additionally included, in any combination, to produce additional embodiments. For example, in one aspect, the housing may be configured to attach to the body at a body location selected from the group consisting of the head, chest, back, arm, and waist. In another aspect, the blower is configured to deliver the pressurized air when the air inlet on a first side surface of the two distinct side surfaces is fully obstructed and the air inlet on a second side surface of the two distinct side surfaces is partially obstructed. In still another aspect, the two distinct side surfaces may comprise two parallel surfaces of the housing, each parallel surface comprising an elongate grate that forms the respective air inlet. In yet another aspect, the apparatus may further comprise a third side surface connecting the two parallel surfaces of the housing, the third side surface also comprising an elongate grate forming an air inlet. In another aspect, the housing may be configured to attach to a head of the user, wherein the housing comprises a lower surface curved to conform substantially in shape to a shape of a surface of the head to which the housing is configured to attach. In still yet another aspect, the housing may be configured to attach to a head of the user, wherein the housing comprises a pad having a shape that corresponds, or is deformable to correspond, to a shape of a surface of the head to which the housing is configured to attach. In still yet another aspect, the pad may comprise a biocompatible visco-elastic foam material and/or a friction-enhancing coating. In another aspect, the apparatus may further comprise a filter element located within the housing proximate the air inlets. In yet another aspect, the apparatus may further comprise a strapping system configured to secure the housing relative to the user's body.

In another embodiment, a positive airway pressure therapy apparatus is provided that includes: a device comprising an housing enclosing a blower, the housing configured to attach to a user's body, wherein the housing may comprise at least two distinct side surfaces, and wherein each side surface defines an air inlet fluidly coupled to the blower; a mask configured to communicate pressurized air generated by the blower to an airway of the user; a delivery tube extending from the housing to the mask, the delivery tube defining an air delivery passage configured to communicate the pressurized air from an outlet of the housing to an inlet of the mask; and a strapping system configured to secure the housing to the body. One or more aspects may be additionally included, in any combination, to produce additional embodiments. For example, in one aspect, the housing may be configured to attach to the body at a body location selected from the group consisting of the head, chest, back, arm, and waist. In another aspect, the blower may be configured to deliver the pressurized air when the air inlet on a first side surface of the two distinct side surfaces is fully obstructed and the air inlet on a second side surface of the two distinct side surfaces is partially obstructed. In yet another aspect, the housing may comprise an outer housing and an inner housing contained within the outer housing, wherein the blower is at least partially supported relative to the inner housing by one or more resilient members. In still yet another aspect, the at least two distinct side surfaces comprise three side surfaces.

In another embodiment, a system for providing positive airway pressure therapy to a user is provided, wherein the system includes: a first mask seal configured to seal to an airway of the user; a shell defining at least part of an air delivery passage configured to deliver pressurized air to the user; and a first adapter positionable between the first mask seal and the shell, the first adapter configured to permit fluid tight attachment of the shell with the first mask seal. One or more aspects may be additionally included, in any combination, to produce additional embodiments. For example, in one aspect, the system may further comprise a second adapter of a con figuration different than the first adapter, the second adapter configured to permit attachment of a second mask seal to the shell, wherein the second mask seal is of a configuration different than the first mask seal. In another aspect, the first adapter and the second adapter each comprise a common universal end configured to attach to the shell. In yet another aspect, the first adapter and the second adapter may comprise a first end configured to receive the first mask seal and the second mask seal, respectively.

In still another embodiment, a strapping system for attaching a positive airway pressure therapy apparatus to a head of a user is provided, the system comprising: an upper strap configured to operatively extend around both a forehead and an occipital bone of the user, the upper strap comprising first and second lower stiffeners configured to be positioned on first and second lateral sides of the head, respectively; first and second upper stiffeners located adjacent the first and second lower stiffeners, respectively; first and second device straps, wherein lower ends of the first and second device straps are attached to the first and second upper stiffeners, respectively, and wherein the first and second device straps are both configured to extend toward a crown of the head; and first and second connectors interposed between the first lower and first upper stiffeners and the second lower and second upper stiffeners, respectively, wherein the first and second connectors are configured to permit movement of the first and second upper stiffeners relative to the first and second lower stiffeners, respectively, in a first direction that is fore-and-aft along a surface of the head, but substantially restrict movement of the first and second upper stiffeners relative to the first and second lower stiffeners, respectively, in a second direction transverse to the first direction. One or more aspects may be additionally included, in any combination, to produce additional embodiments. For example, in one aspect, the system may further comprise a lower strap operable to couple a mask to a face of the user, the lower strap configured to attached to the upper strap near the occipital bone. In another aspect, the lower strap may comprise one or more swivel balls configured to engage a swivel ball retainer formed in the mask. In yet another aspect, the first and second lower stiffeners form discrete components of the upper strap. In yet another aspect, the upper strap further comprises a forehead support. In still yet another aspect, the forehead support may comprise a discrete component to which other sections of the upper strap are removably attached. In another aspect, the forehead support may adjustably support a mask relative to a face of the user. In another aspect, the strapping system may be configured to assist with isolating the mask from movement occurring between one or both of: the first lower and first upper stiffeners; and the second lower and second upper stiffeners. In yet another aspect, each of the first and second device straps may comprise a second end configured to attach to a device located on the crown of the head. In another aspect, the first and second connectors may each permit one or both of: translation of the first and second upper stiffeners relative to the first and second lower stiffeners, respectively; and pivotal movement of the first and second upper stiffeners relative to the first and second lower stiffeners, respectively. In still another aspect, each of the first and second connectors may comprise a fabric member that is rectangular in shape. In yet another aspect, the first and second lower stiffeners and first and second upper stiffeners may each comprise a member formed of a first material, and the first and second connectors each comprise a member formed of a second material different than the first material. In yet another aspect, the first and second connectors may each comprise a pivot configured to permit pivotal movement of the first and second upper stiffeners relative to the first and second lower stiffeners, respectively. In another aspect, the first and second connectors may each comprise a portion of a linear slide, wherein the linear slide is configured to permit translational movement of the first and second upper stiffeners relative to the first and second lower stiffeners, respectively.

In yet another embodiment, a positive pressure airway therapy apparatus for delivering positive pressure therapy to a user's airway may be provided, where the system includes: a device comprising a housing enclosing a blower, the device configured to attach to a crown of the user's head along a median line; a mask defining an outlet configured to fluidly communicate with the airway; a delivery tube extending from an outlet of the device to an inlet of the mask; an upper strap configured to operatively extend around both a forehead and an occipital bone of the user, the upper strap comprising left and right lower stiffeners positionable on left and right sides of the head, respectively; left and right upper stiffeners adjacent the left and right lower stiffeners, respectively; left and right front device straps and left and right rear device straps, wherein: lower ends of the left front and left rear device straps are attached to the left upper stiffener, lower ends of the right front and right rear device straps are attached to the right upper stiffener; and upper ends of the left and right front and left and right rear device straps are attached to the device; and a left movement restrictor interposed between the left lower stiffener and left upper stiffener, and a right movement restrictor interposed between the right lower stiffener and right upper stiffener. One or more aspects may be additionally included, in any combination, to produce additional embodiments. For example, in one aspect, the left and right movement restrictors may be configured to allow movement of the device in a direction along the median line, but substantially restrict movement of the device in a direction transverse to the median line. In another aspect, the upper strap may be configured to secure a forehead support to a forehead of the user. In another aspect, the forehead support may adjustably support the mask relative to the user. In yet another aspect, the system may be configured to isolate movement of the device from the mask.

In another embodiment, a method for dehumidifying a positive airway pressure therapy apparatus is provided, the method comprising: detecting therapy conclusion; and activating a drying mode of the apparatus, wherein the apparatus comprises: a blower; a mask configured to removably attach to a user's head; and a delivery tube defining an air delivery passage fluidly connecting an outlet of the blower to an inlet of the mask. The method further includes: powering the blower after therapy conclusion to provide a flow of air at the outlet; passing the flow of air through the delivery tube and mask; and terminating blower power upon completion of the drying mode. One or more aspects may be additionally included, in any combination, to produce additional embodiments. For example, in one aspect, terminating the blower power may automatically occur at a predetermined time interval after activating the drying mode. In another aspect, detecting therapy conclusion comprises detecting when a portion of the apparatus is removed from the user. In another aspect, the blower may be physically attachable to the user, and detecting when the portion of the apparatus is removed comprises detecting when a housing containing the blower is removed from the user. In yet another aspect, detecting therapy conclusion may comprise detecting the cessation of one or both of inhalation and exhalation. In still another aspect, terminating blower power upon completion of the drying mode may comprise determining when at least one of the delivery tube and mask have reached a predetermined humidity level using one or more humidity sensors.

In still another embodiment, a positive airway pressure therapy apparatus is provided comprising: a device comprising a housing defining an airflow path having an air inlet and an air outlet, wherein the housing comprises a blower for drawing ambient air in through the air inlet and expelling pressurized air via the air outlet, the housing attachable to a body of a user; a mask configured to communicate the pressurized air produced by the blower to an airway of the user; and a delivery tube comprising: a first end fluidly connected to the air outlet; and a second end configured to attach to an inlet of the mask; wherein the housing defines a first noise attenuation chamber between the air inlet and the blower, and a second noise attenuation chamber between the blower and the air outlet. One or more aspects may be additionally included, in any combination, to produce additional embodiments. For example, in one aspect, one or both of the first and second noise attenuation chambers are defined, at least in part, by a material configured to absorb sound. In another aspect, the apparatus may further comprise filter media located in or near the second noise attenuation chamber. In yet another aspect, a pressure sensing passage extends through the second noise attenuation chamber and the filter media and is fluidly sealed from the second noise attenuation chamber. In still another aspect, the filter media is configured to abate noise. In yet another aspect, the housing may comprise: an outer housing; and an inner housing contained within the outer housing. In another aspect, the apparatus may further include a resilient boot supporting the blower relative to the housing, wherein the resilient boot forms a portion of the airflow path within the housing. In still another aspect, the second noise attenuation chamber and the air outlet of the housing are defined at least in part by a nose cone removable attached to the housing. In still yet another aspect, the apparatus further comprises a heat moisture exchange element positioned within an air delivery passage formed between the blower outlet and an outlet of the mask. In another aspect, the apparatus may further include a conduit operatively connected to the blower and configured to deliver cooling air generated by the blower to electronics contained within an electronics cavity of the housing.

In yet another embodiment, a positive airway pressure therapy apparatus is provided comprising: a device comprising a housing defining an airflow path having an air inlet and an air outlet, wherein the housing comprises a blower for drawing ambient air in through the air inlet and expelling pressurized air via the air outlet, the housing attachable to a body of a user; a mask configured to communicate the pressurized air produced by the blower to an airway of the user; and a delivery tube. The delivery tube may comprise: a first end fluidly connected to the air outlet; and a second end configured to attach to an inlet of the mask, wherein the delivery tube defines an air delivery passage configured to deliver the pressurized air to the mask. The apparatus may also include an inner tube located within the delivery tube, wherein at least one end of the inner tube comprises a normally closed passive valve. One or more aspects may be additionally included, in any combination, to produce additional embodiments. For example, in one aspect, the passive valve comprises a membrane attached to a wall of the inner tube at or near the at least one end, the membrane divided into segments that, in a first position, occlude the at least one end. In another aspect, the segments may comprise pie-shaped segments. In yet another aspect, the segments may, upon insertion of a nipple into the at least one end, deflect to a second position to permit entry of the nipple into the inner tube.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE VIEWS OF THE DRAWING

FIG. 1 is a side view of a positive airway pressure therapy apparatus removably secured to a head of a user, according to an exemplary embodiment.

FIG. 2 is a side view of another positive airway pressure therapy apparatus removably secured to the head of the user, according to another exemplary embodiment.

FIG. 3 is a side view of yet another positive airway pressure therapy apparatus removably secured to the head of the user with a strapping system, according to yet another embodiment of the invention.

FIG. 4 is an exploded view of a device (blower housing) and portions therein of the positive airway pressure therapy apparatus shown in FIG. 3, according to an exemplary embodiment.

FIG. 5 is a top view of the exemplary strapping system of FIG. 3, the system shown as laid flat before attachment to the head and to other portions of the positive airway pressure therapy apparatus.

FIG. 6 is a partial side view of a portion of a strapping system in accordance with an alternate embodiment of the invention.

FIG. 7 is a partial side view of a portion of a strapping system in accordance with yet another embodiment of the invention.

FIG. 8 is a cut-away side view of an exemplary delivery tube and face mask of the positive airway pressure therapy apparatus of FIG. 3, according to an exemplary embodiment.

FIG. 9 is a perspective view of a delivery tube and face mask like that of FIG. 8, according to another exemplary embodiment.

FIG. 10 is a side view of the device housing of FIG. 4 with an upper outer housing and nose cone removed, according to an exemplary embodiment.

FIG. 11 is a top view of the housing as shown in FIG. 10.

FIG. 12 is a rear view of a nose cone forming a noise attenuation chamber associated with the housing of a positive airway pressure therapy apparatus, according to an exemplary embodiment.

FIG. 13 is a side view of the nose cone and noise attenuation chamber of FIG. 12, according to an exemplary embodiment.

FIG. 14 is a perspective view of a noise attenuation chamber for use with the positive airway pressure therapy apparatus in accordance with an alternative embodiment of the invention.

FIG. 15 is a schematic diagram of a positive airway pressure therapy apparatus in accordance with an exemplary embodiment of the invention.

FIG. 16 is a diagrammatic view of a positive airway pressure therapy apparatus attached to other portions of the user's body, e.g., the chest or waist, in accordance with embodiments of the invention.

FIG. 17 is a diagrammatic view of a positive airway pressure therapy apparatus attached to yet other portions of the user's body, e.g., the back or arm, in accordance with other embodiments of the invention.

FIG. 18 is an enlarged diagrammatic perspective view of a portion of the device of FIG. 4 in accordance with one embodiment of the invention.

FIG. 19 is an enlarged diagrammatic perspective view of a portion of the delivery tube of FIG. 8 in accordance with one embodiment of the invention.

All figures are illustrated for ease of explanation of the basic teachings of various embodiments of the present invention only; the extensions of the figures with respect to number, position, relationship and dimensions of the parts to form the embodiment may be explained or will be within the skill of one in the art after the following description has been read and understood. Further, the exact dimensions and dimensional proportions to conform to specific force, weight, strength, flow, and similar requirements will likewise be within the skill of one in the art after the following description has been read and understood. Moreover, the figures may, for clarity of description, illustrate environment and other aspects/features that may not form part of the actual claimed invention.

Where used in various figures of the drawing, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when “medial,” “median,” “lateral,” “superior,” “inferior” and similar terms are used, particularly when used to describe relative positions, the terms should be understood to reference the structures shown in the drawing as they will typically be implemented by a vertically-oriented user wearing the apparatus in accordance with embodiments the present invention.

Moreover, the suffixes “a” and “b” may be used with reference numerals throughout this description to denote various right- and left-side parts/features, respectively. However, in most pertinent respects, the parts/features denoted with “a” and “b” suffixes are substantially identical to, or mirror images of, one another. It is understood that, unless otherwise noted, the description of an individual part/feature (e.g., part/feature identified with an “a” suffix) also applies to the corresponding part/feature (e.g., part/feature identified with a “b” suffix). Similarly, the description of a part/feature identified with no suffix may apply, unless noted otherwise, to both the corresponding left and right part/feature.

Furthermore, certain structure e.g., various fasteners, tubing, bearings, electrical components (including but not limited to: wiring, cables, etc.), may be shown diagrammatically or removed from some or all of the views to better illustrate aspects of the depicted embodiments, or where inclusion of such structure/components is not necessary to an understanding of the various embodiments of the invention. The lack of illustration/description of such structure/components in a particular figure, however, is not to be interpreted as limiting the scope of the invention in any way.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

As used herein, the term “face” refers to the front part of a user's head extending from the eyebrows to the chin. “Crown” is used herein to refer to the upper or top area of the head (assuming a standing position). The term “forehead” may be used to indicate the area of the head between the eyebrows and the crown. The term “scalp” may refer to the area bordered by the face anteriorly and the neck to the sides and posteriorly, i.e., that part of the user's head (including the forehead and the crown) that does not include the face. “Median plane” refers to a vertical plane passing through a midline of the head (passing along the sagittal suture) that bisects the head into symmetrical left and right sides. “Median line” refers to the intersection of the median plane with a surface of the head. “Fore-and-aft” may refer herein to a direction along a surface of the head that extends primarily between the face and the back of the head, e.g., along the median line, or otherwise at least extends in this primary direction when viewed perpendicularly to the median plane. “Air” is used herein to denote any fluid to be delivered to a user and may include atmospheric gas, oxygen, other therapeutic gases, and combinations thereof, as well fluids that provide therapeutic and/or other benefits (e.g., water (in liquid or vapor form) for humidification).

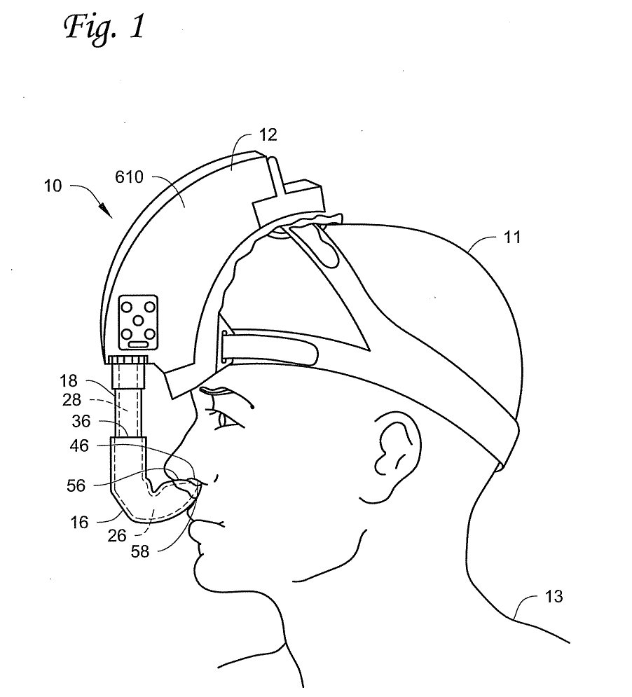

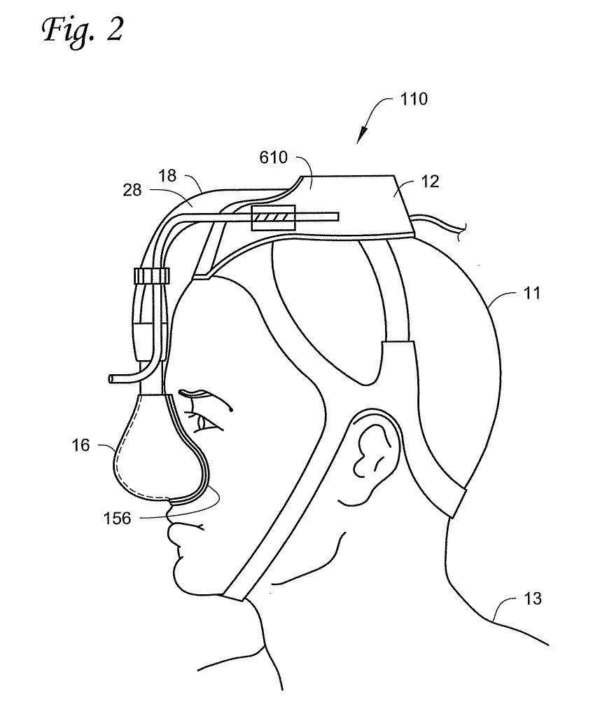

FIG. 1 is a side view of a positive airway pressure therapy apparatus 10 removably secured to a head 11 of a body of a human user 13, according to an exemplary embodiment. FIG. 2 is a side view of another positive airway pressure therapy apparatus 110, also removably secured to the head 11 of the user 13, in accordance with yet another exemplary embodiment. Apparatus 10 and 110 (and others as described herein) may be used for the treatment of, among other ailments, obstructive sleep apnea, asthma, bronchitis, chronic obstructive pulmonary disease (COPD), snoring, and congestive heart failure. These therapies typically pressurize the airway of a user to a pressure in the range of 4-30 cm of water.

One difference between the positive airway pressure therapy apparatus 10 and the positive airway pressure therapy apparatus 110 is the position on the head of the user. For instance, a portion of the positive airway pressure therapy apparatus 10 is designed to be removably secured to at least a portion of the forehead of the user, while a portion of the positive airway pressure apparatus 110 is designed to be removably secured substantially to a crown of the head of the user. While the positive airway pressure therapy apparatus 10 and the positive airway pressure therapy apparatus 110 are generally configured to be secured relative to the forehead and/or crown of the user, other configurations may secure the apparatus to other areas of the scalp or even to other areas of the human body (e.g., the apparatus (or a portion thereof such as a blower housing) may be configured to attach to the user's body at a body location selected from the group consisting of the head, chest, back, arm, and waist). Moreover, while described herein in the context of body-mounted apparatus, those of skill in the art will appreciate that embodiments of the present invention may find application in other conventional (e.g., remotely-located) pressure therapy devices.

The positive airway pressure therapy apparatus 10 and the positive airway pressure therapy apparatus 110 have many common components. For example, the positive airway pressure therapy apparatus 10 and the positive airway pressure therapy apparatus 110 may both include at least a housing 12, a blower 610 (contained within the housing 12 and of which an exemplary embodiment is described in more detail below), and a mask 16. As indicated in the figures, the housing 12 may be configured to secure to the head 11 of the user 13. In certain aspects, the housing 12 may be configured to be secured anywhere on the scalp, e.g., on one or both of the crown (FIG. 2) or the forehead (FIG. 1) of the user, or to other areas of the body.

The blower 610 may be secured on or in the housing 12. The blower 610 is configured to be in fluid communication with the mask 16 by way of a delivery tube 18. The blower 610 may generate a flow of pressurized air and/or other gas that is delivered through the delivery tube 18 and into the mask 16. The flow of pressurized air is directed through an air delivery passage 28 to the mask 16. The air delivery passage 28 may be defined, at least in part, by the housing 12, the delivery tube 18, and/or a portion of the mask 16. The mask 16 is configured to communicate or deliver the pressurized air to an airway 58 (e.g., nasal and/or mouth passages) of the user by directing the pressurized air from the air delivery passage 28 into the airway.

The mask 16 is secured over one or both of a mouth and a nose of the user 13. For example, a seal or other portion of the mask 16 may engage or cover the patient's nose or a portion of the nose. In FIG. 1, the mask 16 includes nares seals which seal to nares or nose passages of the user. This mask 16 is generally referred to as a nares seal. In FIG. 2, the mask 16 covers the nose and sealingly engages an area of the user's face proximate the nose. This mask 16 is generally referred to as a nasal or nose mask. It should be noted that the mask 16 can be configured in any number of ways. In addition to the mask 16 configured with nares seals and the mask configured with a nose mask, the mask 16 can also be configured with a face mask (covering both the nose and the mouth), a mouth seal, or the like.

FIG. 1 shows that the mask 16 generally defines a mask passage 26 having a mask inlet 36, and one or more mask outlets 46 configured to fluidly communicate with the airway 58 of the user 13 (when the mask is positioned to engage the nares of the user). The one or more mask outlets 46 may be surrounded by one or more nares seals 56 that form pillows to engage with and seal to the nares of the user. The nares seals 56 may be removable and interchangeable to permit proper fitting and/or replacement of worn seals 56. In certain aspects, the mask outlet 46 and the seals 56 may be formned as a unitary structure. The mask passage 26 is defined by the mask 16 and is configured to receive pressurized air from the blower 610 through the mask inlet 36. The mask inlet 36 may communicate with the air delivery passage 28 to receive the pressurized air from the blower 610 of the housing 12.

The nasal mask, as shown in FIG. 2, includes a seal 156 that engages the face of the user proximate the nose. The seal 156 may include a portion which passes between the user's mouth and nose as shown. Put another way, the seal 156 may seal around the nose but not the mouth.

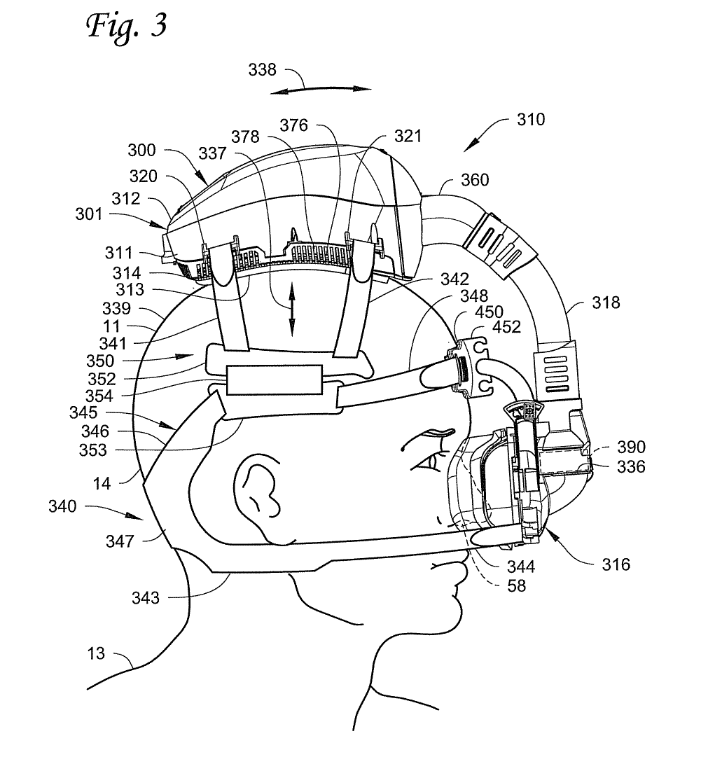

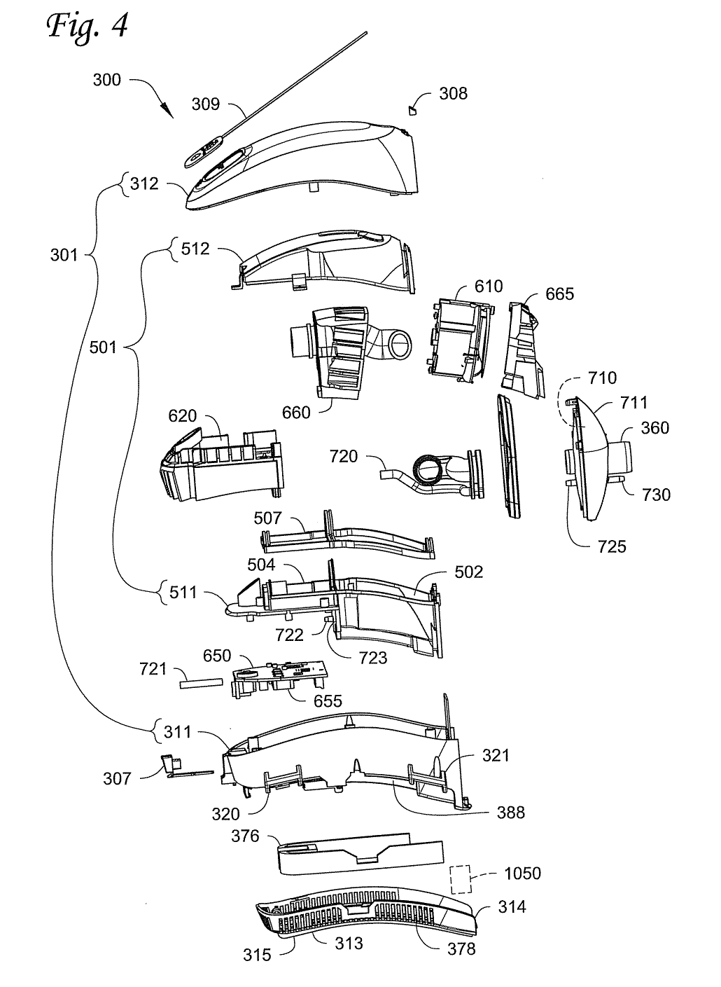

FIG. 3 is a side view of another positive airway pressure therapy apparatus 310 according to another exemplary embodiment of the present invention. Like the apparatus 10 and 110, the positive airway pressure therapy apparatus 310 is designed to deliver a positive pressure therapy to a user's airway, and includes: a device 300 (e.g., including an outer housing 301 and an inner housing 501 (shown in FIG. 4)); a blower 610 (shown in FIGS. 4 and 11) enclosed or contained within the outer housing for generating pressurized air, a mask 316 configured to communicate the pressurized air to an airway of the user; a delivery tube 318 extending from an outlet of the device housing to an inlet of the mask; and a strapping system 340, the latter for attaching the positive airway pressure therapy apparatus, or portions of the apparatus, to the body (e.g., head) of the human user 13 during use. In the illustrated embodiment, the device 300 (e.g., the outer housing 301) is configured to attach to the body of the user, and in one embodiment, to the crown of the user's head 11 along a median line 339 (see FIGS. 3 and 11).

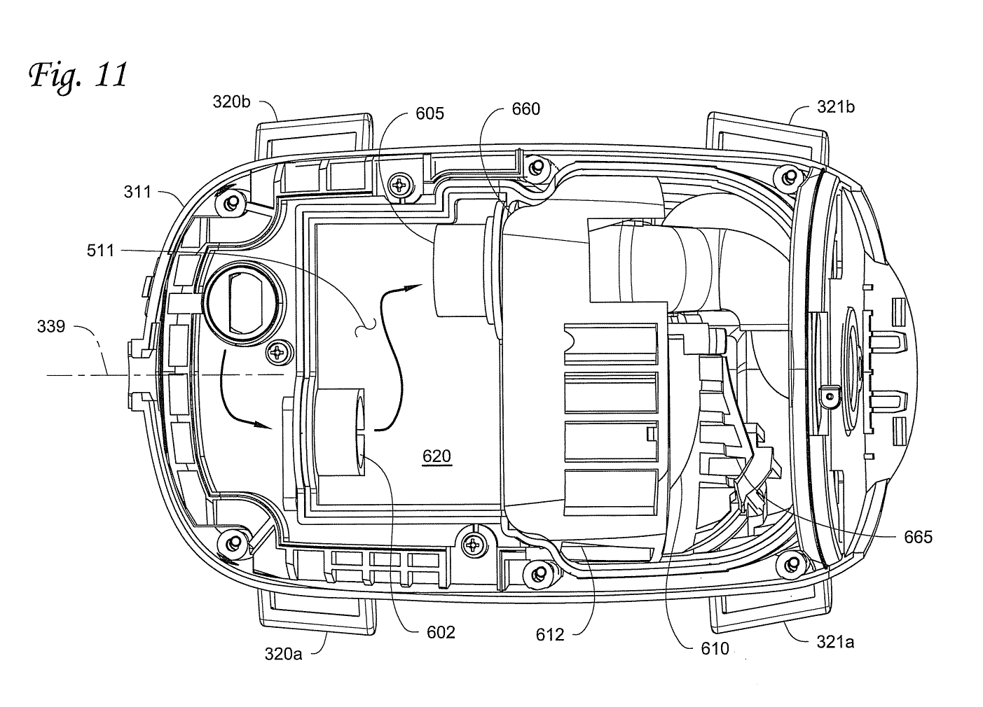

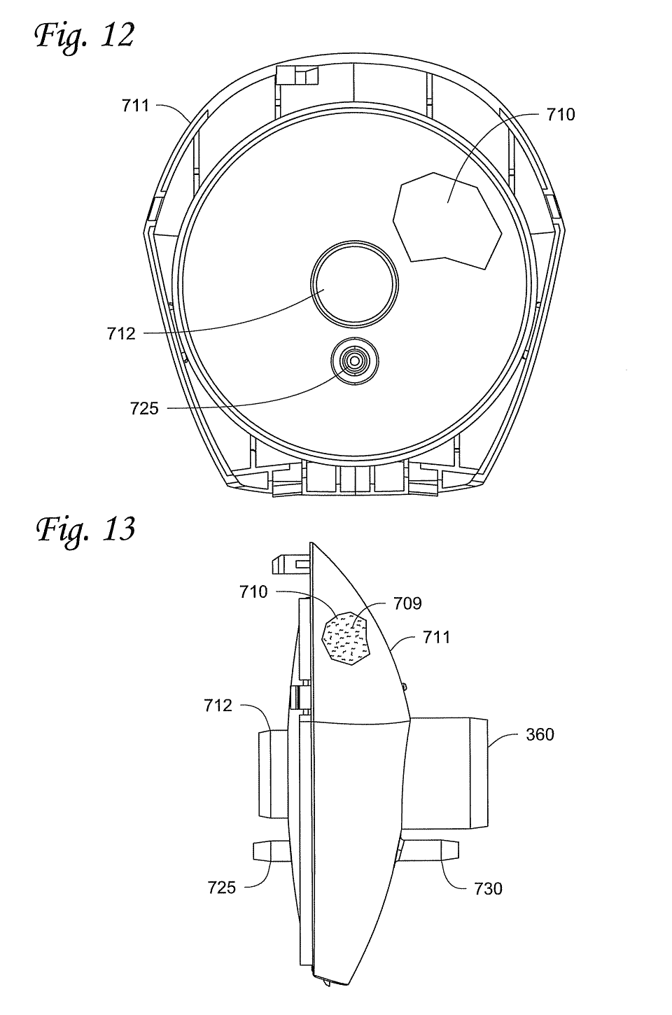

FIG. 4 is an exploded view of a portion of the positive airway pressure therapy apparatus 310 that is contained, or substantially contained, within the outer housing 301 (of the device 300) of the apparatus of FIG. 3, according to an exemplary embodiment. As shown in FIG. 4, the outer housing 301 may include a lower outer housing 311 and an upper outer housing 312. The outer housing 301 (e.g., the upper outer housing 312) may incorporate controls such as an on/off switch panel 309 accessible by the operator to permit control of the apparatus. The exemplary outer housing 301 may also include a nose cone 711 that encloses a noise attenuation chamber 710 (also shown in FIG. 12). In the illustrated embodiment, the nose cone 711 may be removably attached to the outer housing 301 with a lock member 308. An outlet 360 of the noise attenuation chamber 710, which is located on the nose cone 711, may also be referred to herein as the outlet of the device 300, blower 610, and/or outer housing 301.

Contained within the outer housing 301 is an inner housing 501 which may include both a lower inner housing 511 and an upper inner housing 512. An inner enclosure seal 507 may seal the lower inner housing 511 to the upper inner housing 512. The blower 610 may fit within a cavity 502 formed by portions of the inner housing 501.

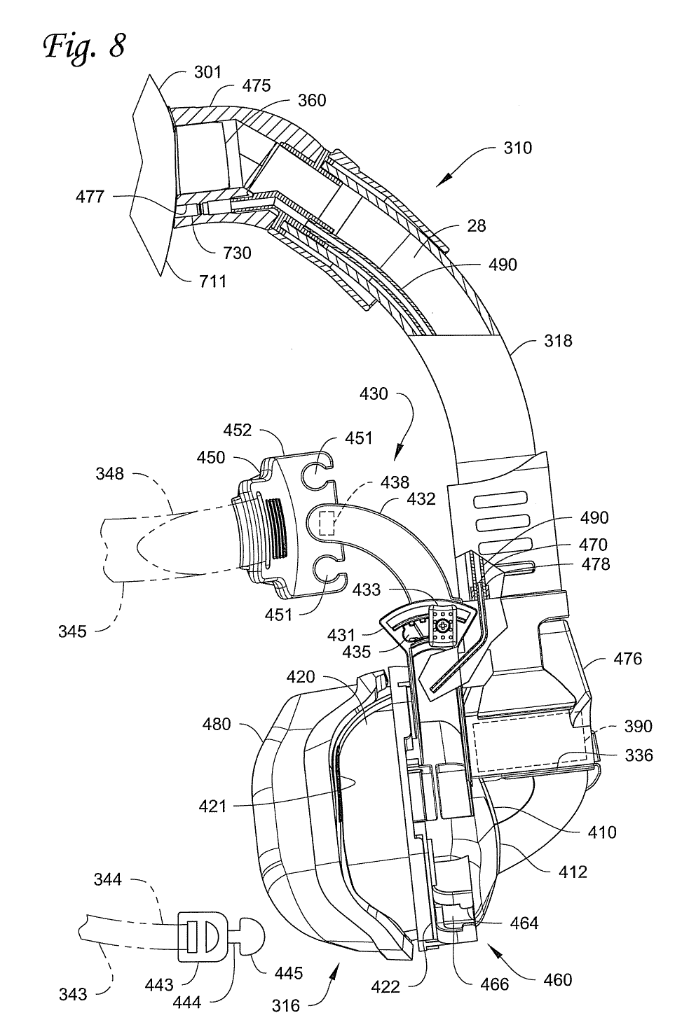

In the illustrated embodiment, the blower 610 may be at least partially surrounded and/or supported relative to the inner housing by one or more resilient members, e.g., a first boot or boot portion 660 and a second boot or boot portion 665 that each defines a cup or recess configured to receive therein respective portions of the blower. The first and second boots 660, 665 may be made from a flexible material that can dampen vibrations that may otherwise result in discomfort and noise to the user. The cavity 502 is sized to accommodate the blower 610 as well as the first boot 660 and the second boot 665. While described and illustrated as incorporating two boots, other embodiments may use a single boot or a plurality of boots for structurally isolating the blower 610 and for dampening vibrations resulting from operation of the blower 610. It should be noted that boots 665 and 660 may form portions of an airflow path (a primary or therapeutic airflow path) extending from an air inlet of the housing (see grate 378 described below) to the outlet 360 through the outer housing 301/inner housing 501 of the device. For instance, boot 660 may faun part of the airflow path connected to or adjacent the inlet of the blower 610. Similarly, boot 665 may form part of the airflow path connected to or adjacent the outlet 360 of the blower 610 (e.g., outlet of the outer housing 301), which connects to an inlet to the delivery tube 318 (shown in FIGS. 3 and 8).

A tube 720 may provide fluid communication (e.g., to measure mask air pressure) of air pressure between a nipple 725 passing through the chamber 710 and a nipple 722 on the lower inner housing 511. The nipple 725 may connect directly to an external nipple 730 located on the outside of the outer housing 301 (which may, as described below, connect to a pressure port in the mask) and further be isolated from other airflow components within the inner/outer housing. An additional tube 721 may provide fluid communication between the nipple 722 and a pressure transducer 655 located on a circuit board 650 that is contained within an electronics cavity defined by the inner and outer housings. In the illustrated embodiment, the electronics cavity is isolated from the primary or therapeutic airflow path (i.e., isolated from the air that is ultimately delivered to the user). It should be noted that the boot or boots 660, 665 can partially enclose or totally enclose the blower 610. Of course, if totally enclosed, provisions may be made for ensuring adequate cooling of the blower 610.

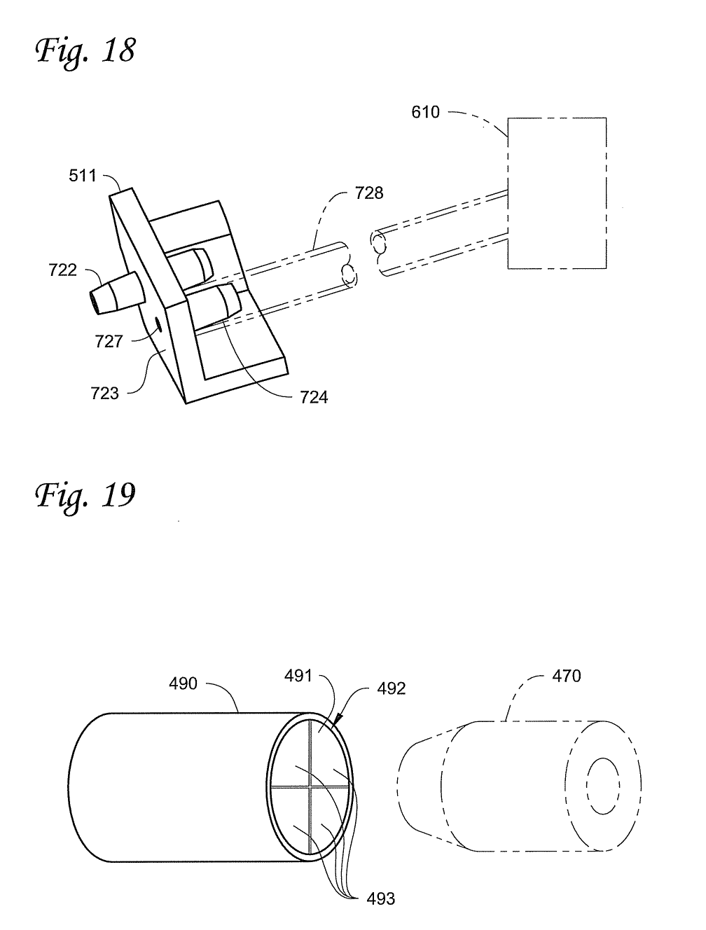

The nipple 722 may pass through a wall 723 of the lower inner housing 511 as also shown in FIG. 18. A nipple 724 (not visible in FIG. 4) may be located on a side of the wall 723 opposite the nipple 722. The nipple 724 may be in fluid communication with an opening 727 passing through the wall 723 into the electronics cavity enclosing the circuit board 650 (see FIG. 4). A first end of a tube or conduit 728 may connect to the nipple 724, while a second end of the conduit 728 is operatively connected to the blower 610, e.g., connected to a port fluidly coupled to the output or pressurized air side of the blower 610. As a result, the blower 610, when powered, may provide a secondary cooling airflow that is directed through the conduit 728 and nipple 724/opening 727 and into the electronics cavity. The conduit 728 may thus be configured to deliver cooling air generated by the blower to the electronics (e.g., to the circuit board 650) contained within the electronics cavity of the housing 301. This cooling air may ultimately exit the electronics cavity via various openings (e.g., unsealed openings faulted around one or more electrical (e.g., data and/or power) connectors 307 (see FIG. 4) located on the outer housing 301).

The lower inner housing 511 may also include a deck 504 which, in some embodiments, includes or supports a first acoustic dampening or first noise attenuation chamber 620 (see also FIG. 11). The circuit board 650, which includes some or all of the electronics associated with the positive airway pressure therapy apparatus 310, may be located and connected below the deck 504. In other words, the circuit board 650 may be connected to a bottom portion of the deck 504 that is opposite and below the noise attenuation chamber 620.

The inner housing 501 may include gaskets or mounts (not shown) that isolate the inner housing from the lower outer housing 311 and the upper outer housing 312. At least one or a portion of a gasket may substantially seal the printed circuit board 650 from the airflow path through the outer housing 301/inner housing 501.

The outer housing 301 (e.g., the lower outer housing 311) may include, or is otherwise attached to, a base 314. In one embodiment, at least a lower surface of the base 314 is curved to conform substantially in shape to a shape of a surface of the body (e.g., a surface of the crown of the head) to which the housing/base is intended to removably mount. As a result, the outer housing 301 may be comfortably attached to the head of the user. A conformable pad 313 may be attached to a lower portion or side of the base 314. The pad 313 may be formed of a material that, in addition to or instead of the base, has a shape that corresponds, or is deformable to correspond, to a shape of a surface of the head to which the housing is configured to attach. The pad 313 may be made of a biocompatible, visco-elastic, foam material such as memory foam so that it easily contours to the head and returns to its original shape after removal.

“Memory foam,” as used herein, refers to any number of visco-elastic, compliant materials that slowly deform to take on or mold to a shape against which they are pressed (e.g., the head), but slowly return to their undeflected shape once the load is removed. Exemplary memory foams may include polyurethane foams such as those foams used in mattresses. The pad 313 (or portions of the pad that contact the user's head) may, in some embodiments, also include a material or coating that inhibits or somewhat restricts movement of the pad relative to the head. For example, the contact surface of the pad can be made of a tacky material or include a friction-enhancing coating 315 that provides a high coefficient of friction relative to skin and/or hair. Of course, it is desirable that the pad 313 be constructed of a material that is also comfortable for the user.

A filter element 376 (e.g., foam media) may fit within the base 314 and thus be positioned below (or in the lower portion of) the outer housing 301. The filter element 376 may be cleanable and/or replaceable by the user. In one embodiment, a gasket or boot may be provided between the base 314 and the lower outer housing 311. The gasket can be any type of biocompatible elastomeric material such as silicone rubber. One or more grates 378 each forming a plurality of slits may be formed in the base 314 to provide an air inlet fluidly coupled to the blower 610 (e.g., connected such that air may flow from the air inlet to the blower). That is, each grate may allow fluid (air) to flow from outside the outer housing 301 to the inside through the filter element 376, whereby it enters the airflow path through the outer housing 301/inner housing 501. In the illustrated embodiment, a grate 378 may be formed on at least two distinct sides of the outer housing 301 (base 314) as shown. Accordingly, the blower 610 may continue to operate even when most of the air inlet slits (e.g., a grate 378) on all sides are completely obstructed. That is, the blower 610 may continue to deliver pressurized air (at pressure and flow levels adequate to provide the intended positive airway pressure therapy to the user) even when the air inlet (grate 378) on a first side of the base 314 is fully obstructed (e.g., by bedding or the like) and the air inlet on the second side surface of the base is partially obstructed. Rather, as long as at least some slits (formed by one of the grates 378) on at least one side of the base 314 are unobstructed, blower operation may be maintained.

In the illustrated embodiment, the filter element may be located within the housing proximate the air inlets (grates 378) such that a gap or space may exist between the grates 378 and adjacent surfaces of the filter element 376. Stated alternatively, the outer peripheral surface of the filter element 376 may be spaced-apart from the inner surface of that portion of the base 314 that defines the grates 378. The gap may allow air drawn in through one area of one grate 378 to flow, within the gap, around the filter element 376 (e.g., to an area of the filter element proximate one of the other grates) before passing through the filter element. Such a configuration may be beneficial, for example, when a portion of one of the grates is occluded, or when a portion of the filter element becomes obstructed (e.g., by dust or the like).

In the illustrated embodiment, the two distinct side surfaces may be formed by two parallel or opposite (e.g., lateral side) surfaces of the outer housing 301, wherein each parallel surface has an elongate grate 378 that forms the respective air inlet. In other embodiments, a third side (e.g., rear side as shown in FIG. 3) surface connecting the two parallel side surfaces of the housing 301 may also include an elongate grate 378 forming another air inlet.

The positive airway pressure therapy apparatus 310 may also include the delivery tube 318 (shown in FIG. 3) having a first end that is attached and fluidly connected to the air outlet 360 of the blower 610/outer housing 301, and a second end configured to attach to an inlet 336 (see also FIG. 3) of the mask 316. The blower 610 may draw ambient air from the atmosphere in through the air inlet (grate 378) and through the filter element 376 held by the base 314, and expel pressurized air via the air outlet 360 to the tube 318 and mask 316.

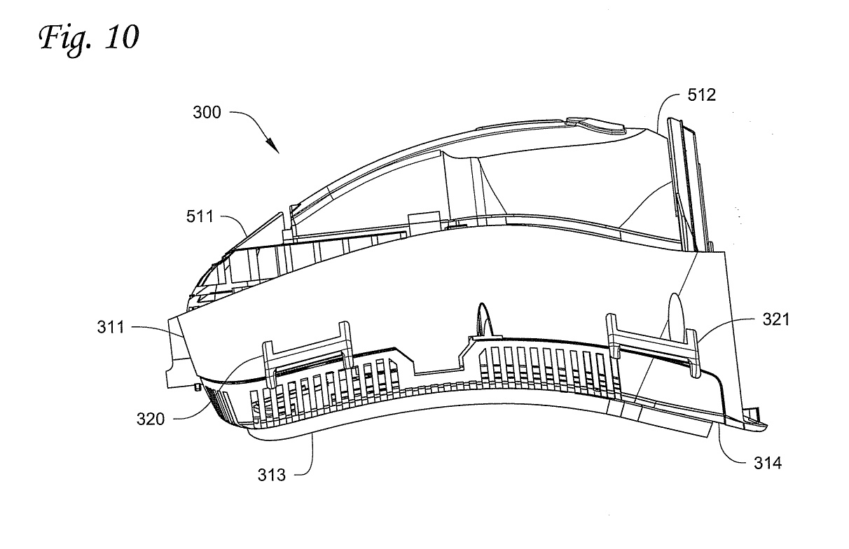

The housing may also be provided with one or more attachment points, which in the illustrated embodiment, are configured as four retainer lugs, such as rear lugs 320 (320a and 320b) and front lugs 321 (321a and 321b). The retainer lugs 320, 321 (shown in FIG. 3, FIG. 4, FIG. 10, and FIG. 11) have slots formed therein and are sized to receive and retain a device strap as further described below. While only two retainer lugs 320 and 321 are shown in FIGS. 3 and 4, similar retainer lugs are positioned on the opposite side of the lower outer housing 311 as shown in FIG. 11.

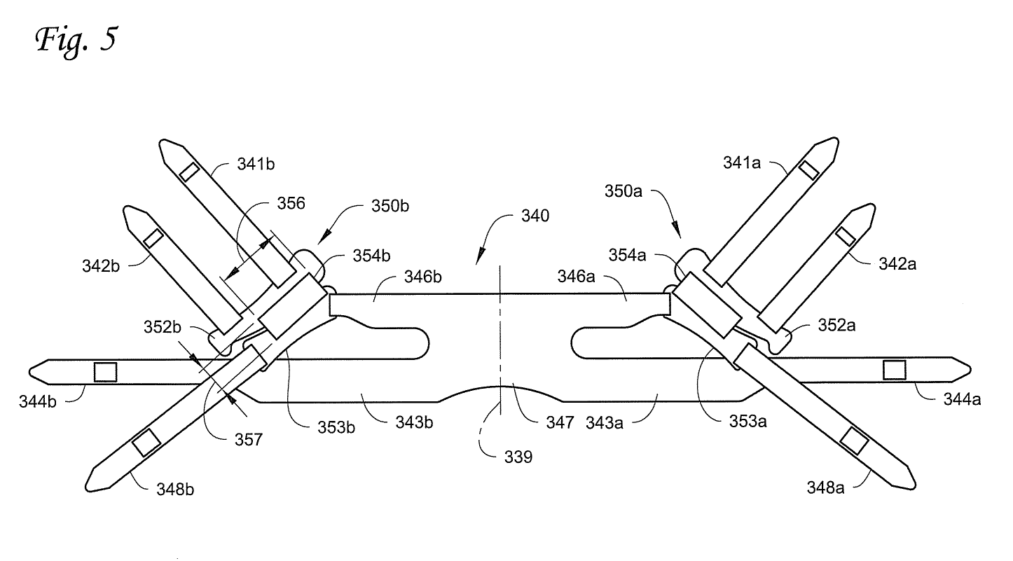

FIG. 5 is a top plan view of the exemplary strapping system 340 illustrated in FIG. 3, but shown laid flat and before attachment to the user and the other components (e.g., outer housing 301) of the positive airway pressure therapy apparatus 310. With reference to FIGS. 3 and 5, one illustrative strapping system 340 for removably attaching the positive airway pressure therapy apparatus 310 to the head of a user will now be described.

As shown in FIG. 3, the strapping system 340 may hold the outer housing 301 of the device 300 to the head and hold the mask 316 in sealing engagement with the face of the user. As shown in FIGS. 3 and 5, the illustrative strapping system 340 may include one or more, e.g., two, bifurcated movement restrictors 350 (e.g., first (right) movement restrictor 350a, and second (left) movement restrictor 350b). Each bifurcated movement restrictor 350 may include an upper stiffener 352 (first (right) upper stiffener 352a and second (left) upper stiffener 352b) and a lower stiffener 353 (first (right) lower stiffener 353a and second (left) lower stiffener 353b). First (right) and second (left) connectors 354 (354a, 354b) are interposed between the first lower (353a) and first upper stiffeners (352a) and the second lower (353b) and second upper (352b) stiffeners, respectively. In the illustrated embodiment, the first and second (e.g., right and left) lower stiffeners are configured to be positioned on first (e.g., right) and second (e.g., left) lateral sides of the head, respectively, as indicated in FIG. 3. As further illustrated in the figures, the first and second upper stiffeners 352 are located adjacent the first and second lower stiffeners 353, respectively.

As described in more detail below, the first and second connectors are configured to permit movement or translation of the first and second upper stiffeners relative to the first and second lower stiffeners, respectively, in a first direction 338 (see FIG. 3) that is generally fore-and-aft along a surface of the head, but substantially restrict movement of the first and second upper stiffeners relative to the first and second lower stiffeners, respectively, in a second direction 337 along the head surface transverse to the first direction.

First device strap (e.g., right rear device strap 341a and/or right front device strap 342a), and second device strap (e.g., left rear device strap 341b and/or left front device strap 342b) may each include a first or lower end that attaches to its respective first (e.g., right) upper stiffener 352a and second (e.g., left) upper stiffener 352b as shown in FIG. 5. As shown in FIG. 3, each device strap 341, 342 may extend toward the crown of the head, e.g., toward the median line, where respective second or upper ends of each device strap are configured to attach to the outer housing 301 of the device 300 when the device is located on the crown of the head. Front upper straps 348 (348a, 348b) (forehead) and rear upper straps 346 (346a, 346b) may attach to their respective lower stiffeners 353 as also indicated in FIGS. 3 and 5. One or more lower straps 343 may also be connected to the upper strap 345 (e.g., via a web 347 connected to the rear upper strap 346) near the occipital protuberance or bone 14 as shown in FIGS. 3 and 5.

The strapping system 340 is shown in its entirety (and separated from the remainder of the apparatus 310) in FIG. 5. FIG. 3, on the other hand, shows the right side of the strapping system 340 in relation to other elements of the positive airway pressure therapy apparatus 310 as mounted to the head 11 of the user 13. As the exemplary strapping system 340 is symmetrical about a median plane 339 of the head, one side (e.g., the right side shown in FIG. 3 and the side shown with “a” suffixed reference numerals in FIG. 5) will be described in detail with the understanding that the other (e.g., left) side is a mirror image. Once again, where beneficial to an understanding of the invention, the corresponding right- and left-side components of the strap system 340 may be distinguished/identified with “a” and “b” suffixes. For instance, e.g., device straps 341b and 342b (see FIG. 5) are left-side straps that correspond to right-side device straps 341a and 342a, respectively (the straps 341a, 342a are identified generically as device straps 341, 342, respectively, in FIG. 3). Similarly, forehead strap 348b (see FIG. 5) is generally a mirror image of the forehead strap 348a (the latter, once again, illustrated generically as strap 348 in FIG. 3), only located on the left or opposite side of the head. This relationship between right- and left-side components, unless otherwise noted, applies to the other commonly-numbered elements of the system 340 (as well as to other portions of the apparatus 310 where “a” and “b” suffixes are used).

One end of each of the device straps 341, 342 may pass through the retainer lugs 320, 321, respectively, of the outer housing 301 as shown in FIG. 3. The other end of each device strap 341, 342 may attach to the upper stiffener 352 of its respective restrictor 350. Each forehead strap 348 may be attached to a frame 452 of a forehead support 450 (to secure the support to the forehead) at one end while another end of each forehead strap 348 is attached to the lower stiffener 353 of its respective movement restrictor 350. Each lower strap 343 may have one end 344 (344a or 344b in FIG. 5) attachable to one side of the mask 316 to couple the mask to the face of the user.

In the illustrated embodiment, the front upper straps 348, rear upper straps 346, and lower stiffeners 353 are shown and described as discrete components that are attached to one another to form, along with the frame 452 of the forehead support 450, a generally continuous upper strap 345 configured to operatively extend about a circumference of the head, e.g., extend around both the forehead and the occipital bone. While shown as an assembly of these discrete components, an alternative upper strap 345 could be configured as a single unitary strap extending entirely about the head (or at least extending from one side of the frame 452 of the forehead support 450, around the head, to the opposite side of the frame 452). In such an embodiment, the lower stiffeners 353 could be incorporated into the upper strap 345 by configuring areas of the upper strap to have different elasticity (e.g., thicker section) than other portions of the upper strap. Such a configuration would thus still provide the desired functionality of the movement restrictor 350 as further described below.

In one embodiment, the length of one or more straps of the strapping system 340 can be adjusted. For example, each strap end may include a portion of a hook and loop fastener. Positioned along one side of each strap may be another portion of the hook and loop fastener so that the strap end can be threaded through a retainer lug and folded over to engage the other portion of a hook and loop fastener (see e.g., FIG. 5). In this way, a user can adjust the straps to achieve a snug and comfortable fit regardless of variability in head size.

The exemplary strapping system 340 may include the pair of bifurcated movement restrictors 350 (350a, 350b in FIG. 5). Once again, each movement restrictor 350a, 350b is substantially the same. As a result, only one movement restrictor (e.g., 350a) will be described in detail with the understanding that the other movement restrictor (e.g., 350b) is substantially the same in appearance and function.

Each movement restrictor 350 includes the upper stiffener 352, lower stiffener 353, and connector 354. In one embodiment, each connector 354 is made from elastic or an elastomeric material that allows controlled or limited movement between its respective upper stiffener 352 and lower stiffener 353. For example, each connector 354 may be configured to allow some relative movement between its respective stiffeners 352 and 353 in a first direction that is fore-and-aft along the surface of the head, but substantially limit relative movement between each upper stiffener 352 and its associated lower stiffener 353 (and thus between the device and the upper strap 345) in a second direction that is transverse to the fore-and-aft direction.

In the illustrated embodiment, each connector 354 may be formed of a rectangular piece of material (e.g., fabric) having a first side or edge attached to the upper stiffener 352 and a second (e.g., opposite) side or edge attached to the lower stiffener 353. The first and second edges may both form a long side 356 of the rectangle as shown in FIG. 5, while a short side 357 extends between the two stiffeners 352, 353.

In the illustrated embodiment, the lower stiffeners 353 and upper stiffeners 352 each define a member formed of a first material, and the connectors 354 each includes a member formed of a second material different than the first material. In this exemplary embodiment, each connector 354 is made of a material that stretches (or otherwise permits relative movement of the first side of the connector relative to the second side) more easily in the first direction (e.g., fore-and-aft direction 338 in FIG. 3), but has reduced ability to stretch or move in the second direction 337 (e.g., laterally toward and away from the crown of the head in FIG. 3). Due to the rectangular shape of the connectors 354, each connector 354 may also be understood to stretch in a rotational sense when a torque is applied to the connector (e.g., when the device 300 moves along the median line of the head). Put another way, each connector 354 may be compliant in a rotational sense and along its length (e.g., the connectors may permit pivotal movement of the first and second upper stiffeners 352 relative to the first and second lower stiffeners 352, respectively), but have less compliance in a direction transverse to the length of the connector 354 (limited compliance or stretch in a direction transverse to the median line of the head).

As described in more detail below, the first and second connectors are configured to permit movement or translation of the first and second upper stiffeners relative to the first and second lower stiffeners, respectively in a first direction that is fore-and-aft along a surface of the head, but substantially restrict movement of the first and second upper stiffeners relative to the first and second lower stiffeners, respectively, in a second direction transverse to the first direction. Stated alternatively, each (e.g., left and right) movement restrictor 350 may allow some movement of the housing of the device 300, relative to the upper strap 345 (and relative to a surface of the head), in the fore-and-aft direction 338 along the median line, but substantially restrict relative movement of the housing of the device, relative to the upper strap and head, in a direction 337 transverse to the median line, e.g., restrict side-to-side movement of the housing along the head.

Accordingly, when the positive airway pressure therapy apparatus 310 (e.g., the housing 301) is removably attached to the user's head 11, the movement restrictors 350 (right (350a) and left (350b) restrictors in FIG. 5) may inhibit lateral motion of the housing 301 with respect to the head. Thus, the movement restrictors 350 can also be thought of as lateral motion stiffeners in that they restrict motion of the housing 301 from side-to-side. As a result, they may reduce the chances that the housing 301 may fall to the side of the head when in use. However, the stiffeners 350 are more compliant in the anterior-posterior (fore-and-aft) direction, thus allowing some sliding of the housing 301 toward the back (or front) of the head.

The delivery tube 318 may be of sufficient length and constructed of a soft, compliant material so as to allow the housing 301 to move or slide toward the back of the head without imparting a substantial load to the face mask 316 and face of the user. Said differently, the strapping system 340, in conjunction with the material and length of the delivery tube 318, may isolate the mask from movement occurring between one or both of: the first lower and first upper stiffeners; and the second lower and second upper stiffeners. Such isolation may thus substantially decouple or isolate movement of the outer housing 301 of the device 300 from the face mask 316, at least through a limited range of movement of the housing.

One advantage of the strapping system 340 is that there are some positions in which a patient can rest so that the load of the device 300 on the user's head will be substantially lessened. For example, if the user sleeps or rests on his or her back, the bifurcated restrictors 350 (350a, 350b) allow the outer housing 301 of the device 300 to move toward the back of the head of the user while remaining generally centered on the median line of the head. Thus, the device 300 can move until it rests upon a sleeping surface such as a pillow. Once the housing is resting on the sleeping surface, the weight of the device 300 may be at least partially supported by the sleeping surface rather than completely by the user's head.

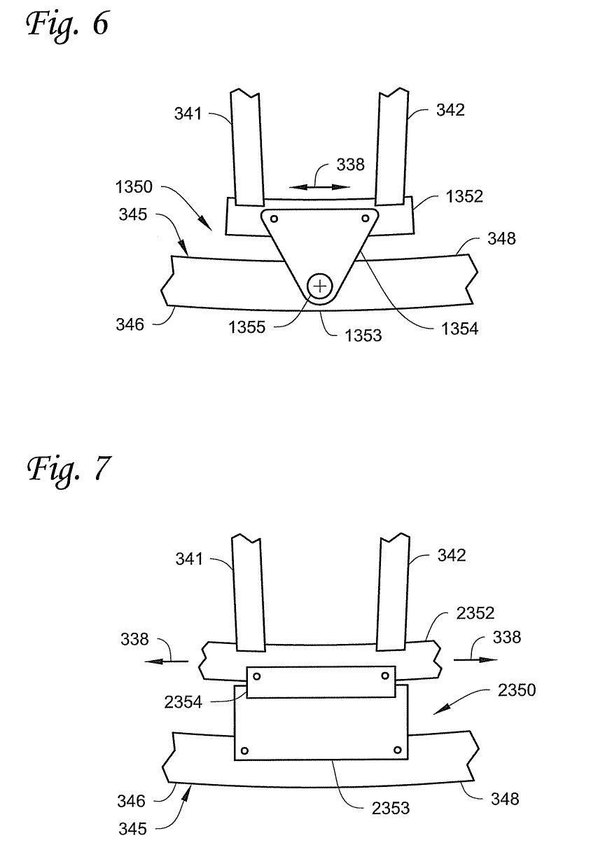

Other movement restrictor configurations may be used to attain equivalent results. As an example, the use of mechanical devices as an alternative to the elastic or stretchy material provided by the restrictor 350/connector 354 are contemplated. For instance, FIG. 6 illustrates a right side portion (left side again being a mirror image) of a strapping system similar in most respects to the strapping system 340 except that it replaces each movement restrictor 350 with a corresponding movement restrictor 1350 incorporating a rotational pivot 1355 on both right and left (not shown) sides of the head. Each restrictor 1350 may include an upper stiffener 1352 and a lower stiffener 1353. In the illustrated embodiment, the lower stiffener 1353 could be configured as a separate component (e.g., like the lower stiffener 353 already described herein), or as a specific, integral area of the upper strap 345 configured to have increased rigidity. For example, the lower stiffener 1353 could merely be a rivet or eyelet in the strap 345 to receive the pivot 1355. The upper stiffener 1352 (attached to device straps 341, 342) may rigidly attach to a rigid connector 1354 forming the pivot 1355, wherein the connector is pivotally attached to the lower stiffener 1353 at the pivot 1355. As a result, the pivot of the connector 1354 allows pivotal movement of the upper stiffener 1352 relative to the lower stiffener 1353. By placing the pivot 1355 on the lower stiffener (upper strap 345) as opposed to the upper stiffener 1352, a larger radial distance may be provided between the pivot and the outer housing 301 (see FIG. 3). As a result, during operation, the rotational movement afforded by the pivot 1355 may provide for movement of the upper stiffener 1352 (and thus the outer housing 301 of the device 300; see FIG. 3) relative to the lower stiffener 1353 in generally the fore-and-aft direction 338, while restricting lateral movement (restricting movement in the transverse direction).

FIG. 7 illustrates yet another embodiment of a movement restrictor 2350 (only right side shown, but left side is, once again, understood to be generally a mirror image) that may replace the movement restrictor 350 of FIGS. 3 and 5, as well as the movement restrictor 1350 of FIG. 6. In this embodiment, the movement restrictor 2350 again includes an upper stiffener 2352 (to which the device straps 341, 342 are secured) and a lower stiffener 2353 (to which the upper strap 345 (e.g., front strap 348) is secured). Each of the upper and lower stiffeners 2352, 2353 may each have attached thereto a separate portion of a linear slide or linear bearing 2354 of the movement restrictor 2350. The linear slide/bearing, as is known in the art, permits translational movement of the upper stiffener relative to the lower stiffener in the fore-and-aft direction 338, but limits corresponding lateral movement.

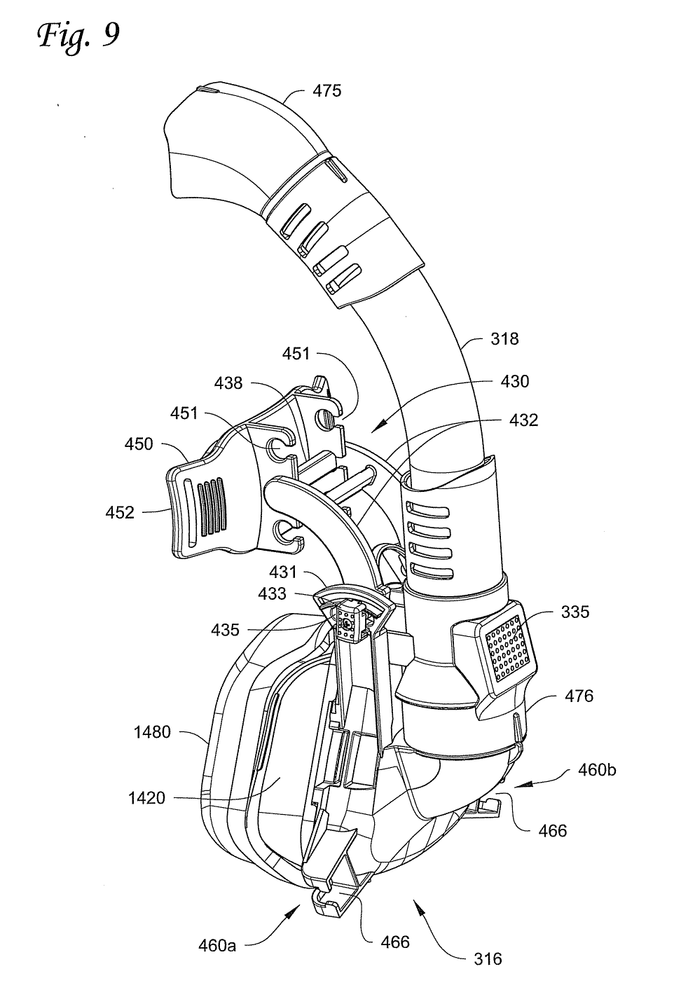

FIG. 8 is an enlarged side view (with some sections cut-away for clarity) of the hose or delivery tube 318 and face mask 316 of the positive airway pressure therapy apparatus 310, according to an exemplary embodiment, while FIG. 9 is a perspective view of the same. The air delivery passage 28 of the delivery tube 318 may be configured to communicate the pressurized air from the outlet 360 of the outer housing 301/blower 610 to the inlet 336 of a mask shell 410 of the mask 316. Tn the illustrated embodiment, the delivery tube 318 also operatively provides independent and separate fluid communication between the nipple 730 on the nose cone 711/second expansion chamber 710 (see also FIG. 4) and a nipple 470 on the shell 410 as shown in FIG. 8. Proximal hose connector 475 of the delivery tube 318 contains a mating port 477 that receives the nipple 730 and seals it from the primary air delivery passage 28 within the delivery tube 318. An inner tube 490 may be located within the delivery tube 318. The tube 490 extends distally from the proximal hose connector 475 to a distal hose connector 476 where it sealably engages a port 478 via the nipple 470 at the shell 410. Such a system may provide sealed fluid communication between the air pressure present within the mask shell 410 and the pressure transducer 655 located on the circuit board 650 (see FIG. 4) through a tube or fluid flow path that includes the pressure transducer 655, nipple 722, pressure tube 720, nipple 725, nipple 730, proximal hose connector 475, tube 490, distal hose connector 476, port 478, and nipple 470 of mask shell 410. In short, a fluid flow path places the pressure transducer 655 in fluid communication with the environment inside the mask 316.

In one embodiment, the inner tube 490 may include a normally closed, passive valve 492 at one or both ends (e.g., referring to FIG. 8, the valves may be incorporated into both: the proximal hose connector 475 near the nipple 730; and into the other end of the tube 490 near the nipple 470). An exemplary passive valve 492 is illustrated diagrammatically in FIG. 19. As shown in this view, each passive valve 492 may be formed by a flexible, thin portion or membrane 491 attached or integrally formed at (or, alternatively, near) the operative ends of the tube 490. Each membrane 491 may be attached to a wall of the inner tube 490 as shown in FIG. 19, and be divided (e.g., slit cut) to form one or more segments, e.g., four pie-shaped segments 493. The segments 493 remain anchored to the wall of the tube 490 near their peripheral edges as shown. In a first or undeflected position, the segments 493 of the valve are nomially closed to occlude at least one (and preferably both) ends of the inner tube. However, the segments 493 of the membrane may permit the respective nipples to push past the membrane 491 by contacting and deflecting the segments 493 to a second position as the nipple is inserted into the inner tube 490. However, when the tube 490 (e.g., the delivery tube 318) is detached from the nipple 470 or 730 (e.g., when the delivery tube 318 is detached from the device 300 and/or the mask 316), the respective segments 493 of the valve/membrane may return to their undeflected (normally closed) positions, generally blocking entry into the tube 490. As a result, when the user washes the delivery tube 318, the segments of the valve 492/membrane 491 may resist the entry of water into the tube 490.

As clearly shown in FIG. 9, the apparatus 310 may also include a vent or vent leak 335 as is generally known in the art to primarily ensure that exhaled carbon dioxide is adequately purged from the system during operation. While the vent leak could be located on the mask shell 410, it is, in the illustrated embodiment, formed along a portion of the delivery tube 318, e.g., at or near the distal hose connector 476.

The mask 316 may include the shell 410 (which defines at least part of the air delivery passage 28 to deliver pressurized air to the user), a mask seal 480 configured to seal to, and fluidly communicate with the airway 58 (see FIG. 3) of the user, and an interchangeable portion or adapter 420. The adapter 420 is positionable between the seal 480 and the shell and is configured to permit fluid tight attachment of the shell with the seal. Accordingly, the adapter 420 has a first end 421 shaped to receive the seal 480, which may be a face mask or other type of seal, and a second or universal end 422 which attaches to the shell 410.

In one embodiment, users have a wide variety in preferences in seals. For example, one user may prefer one type of seal that may seal (or is perceived to seal) more effectively than another, or that may be more comfortable to wear. The above-described system allows users to select their preferred mask seal, or to try different seals, without concern for whether the seal was specifically manufactured for use with the mask shell 410. For instance, a first adapter 420 may be utilized with a first seal 480 to operatively connect the first seal to the shell 410 as shown in FIG. 8. Moreover, a second adapter (e.g., adapter 1420 of FIG. 9) of a configuration different than the first adapter 420 may also be provided. The second adapter 1420 may be configured to permit attachment of a second mask seal (e.g., seal 1480 of FIG. 9) to the shell 410, wherein the second mask seal is of a configuration different than the first mask seal. That is to say, the first adapter 420 and the second adapter 1420 may each include a first end configured to receive the first mask seal 480 and the second mask seal 1480, respectively. These first ends may thus be of different configurations to accommodate attachment of their respective seals. However, the first adapter 420 and the second adapter 1420 may each comprise a common (e.g., identical) second or universal end (see e.g., end 422 in FIG. 8) so that the adapters are both configured to attach to the same shell 410. Stated alternatively, the universal end of each adapter is similar in configuration to permit airtight attachment of the different adapters to the same shell. While only two different seals and corresponding adapters are illustrated, any number of different seals may be attached to the same shell by providing a corresponding number of adapters specifically designed for each respective seal.

As a result, a manufacturer may provide different adapters to accommodate a variety, e.g., the most popular, of mask seals. The adapter corresponding to a different preferred mask seal can replace the existing adapter. When the preferred mask seal 480 is attached to the first end 421 of the adapter 420, the second end 422 can be sealingly attached to the frame 412 of the mask shell 410. An advantage for a new user of the positive airway pressure therapy apparatus 310 is that it can accommodate the mask seal that the user may already prefer. This may lower the resistance of the user to changing from a previous device to a different positive airway pressure therapy apparatus.

As shown in FIGS. 8 and 9, the shell 410 may also include a frame 412 to which is attached a forehead support adjustment system 430. The forehead support adjustment system includes a first beam 431 attached to the frame 412 of the shell 410, and a second beam 432. As indicated in FIG. 9, a first and second beam may be located on each of right and left sides of the system 430. The first beam 431 may include an opening 435 through which projection portions 433 formed at a first (e.g. lower) end of the second beam 432 are slidably received. The second beam 432, therefore, may slide within the opening 435 to provide adjustment of the forehead support 450 with respect to the user's forehead. Stated another way, the sliding of the projection portions 433 with respect to the openings 435 allows the user to adjust or alter the proximity of the forehead support 450 to the user's forehead. In the illustrated embodiment, the projection portions 433 may engage the opening 435 such that they may lock the first beam 431 relative to the second beam 432 once the two beams are adequately adjusted. Accordingly, the forehead support may adjustably support the mask 316 relative to the face.

The forehead support 450 includes, in the illustrated embodiment, the forehead support frame 452, which is held in place by ends of the front upper straps 348 (see FIGS. 3 and 8). The forehead support frame 452 may include an element that receives a bar 438 formed at a second (upper) end of the second beam 432 (see FIG. 9). In the illustrated embodiment, the frame 452 defines one or more openings 451 that may receive the bar 438. The engagement of the bar 438 with one set of the openings 451 allows the frame 452 to move, e.g. pivot, with respect to the second beam 432 and, more particularly, with respect to the bar 438. Once again, this movement accommodates different slants and slopes of the forehead. In the embodiment shown in FIGS. 8 and 9, the frame 412 is provided with multiple openings 451 to receive the bar 438. Providing multiple openings 451 at different elevations on the forehead support frame 452 allows for different placements of the forehead support 450 on the forehead and/or accommodates other anatomical variations (e.g., users with higher or lower foreheads). These adjustments can be easily made by the user to find a comfortable position where the face mask will substantially seal to the face of the user and where the forehead support 450 is placed at a comfortable location on the forehead.

In the illustrated embodiment, the forehead support 450 forms a discrete component to which other sections of the upper strap 345 are removably attached. However, in alternate embodiments wherein the upper strap portions 346, 348 and lower stiffeners 353 form a unitary and continuous circumferential strap 345 about the forehead and the occipital bone, the frame 452 of the support 450 may be attached to the strap 345 via most any conventional method, e.g., hook and loop fastener. In such an embodiment, the forehead support 450 could be aligned as (or otherwise attached to) part of the upper strap 345 directly.

With reference still to FIGS. 8 and 9, the frame 412 of the mask shell 410 may also include a pair of swivel ball retainers 460 that assist, in conjunction with the strap 343 of the strap system 340, in securing the mask 316 to the user's face. As indicated in FIG. 9, one retainer 460 may be provided on each side of the mask 316, both of which are substantially identical to one another.

One or both ends 344 of the lower strap 343 may include a clip 443 having one end with a slot therein adapted to receive the strap 343 and another end forming a stem 444 that carries a swivel ball 445 (e.g., ball forming a full or partial sphere) configured to engage one of the swivel ball retainers 460 of the mask. To permit attachment, each swivel ball retainer 460 has a side with a slot 464 formed therein that allows the stem 444 to pass. Another surface of the swivel ball retainer 460 is sized to receive the ball 445 once the stem 444 has passed through the slot 464. This surface also defines an enlarged opening 466 that allows the stem 444 to move within the opening 466 as the ball 445 swivels or pivots within the opening 466. In this way, the ball and stem of the clip 443 can swivel or move and directly pull on the swivel ball retainer 460 of the mask 316. This allows the clip 443 to move relative to the mask 316 as strapping system adjustments are made while also preventing the clip from projecting outwardly from the lower strap 344. This may provide additional comfort for the user as the clip 443 will not protrude outwardly where it can catch on other items during sleep. The clip 443 may also advantageously avoid a position where a component of the load placed on the face mask 316 might cause discomfort to the user.

FIG. 10 is a side elevation view of the lower outer housing 311 including the base 314 with the upper outer housing 312 removed, according to an exemplary embodiment (while the upper outer housing 312 of the outer housing 301 is not shown in FIG. 10; a complete outer housing 301 is illustrated in FIG. 3). Contained within the outer housing 301 is the inner housing 501 (see FIG. 4), which includes both the lower inner housing 511 and the upper inner housing 512. The lower inner housing 511 may be rigidly fixed to the lower outer housing 311, or secured in position through the use of one or more structures made from compliant materials such a silicone rubber or other materials that can absorb and/or isolate shock, vibration, and noise.

In the embodiment illustrated in FIG. 10, the upper inner housing 512 is rigidly attached to the lower inner housing 511. However, other embodiments could provide a set of silicone seals or other resilient member(s) between the lower and upper inner housings 511 and 512. The set of silicone seals/resilient member(s) could dampen vibrations and reduce transmission of those vibrations (as well as sound) to the outer housing 301. In either configuration, the resulting arrangement (when assembled) is an inner housing 501 contained within an outer housing 301 (see FIG. 3). This “housing within a housing” is useful for reducing noise produced by the positive airway pressure therapy apparatus 310. The positive airway pressure therapy apparatus 310 may also include other noise reducing/abatement features as described elsewhere herein.

FIG. 11 is a top view of the assembled lower outer housing 311 and the lower inner housing 511 when the upper outer housing 312 and upper inner housing 512 are removed, according to an exemplary embodiment. As illustrated in this view, the lower inner housing 511 may receive the blower 610 and, in one embodiment, form at least a part of a first noise attenuation chamber 620 located in the airflow path proximate an intake of the blower 610, e.g., between the air inlet (grate 378 of FIG. 3) and the blower.

The blower 610 may include a blower motor 612 which rotationally powers an impeller for moving fluid along the airflow path through the outer housing 301/inner housing 501. As shown in FIG. 11, the blower 610 and blower motor 612 are substantially encased in the one or more compliant boots 660, 665, which, in the illustrated embodiment, is made of a flexible material such as silicone rubber (or other material that can absorb shock, vibration, and noise). While the illustrated compliant boots (as shown in FIG. 4) include two separate parts, it is noted that they could be made from any number of parts including a single part.

When the blower 610 is in operation, it pulls air or fluid into the outer housing 301 through the grates 378 formed on the base 314, the latter which form the start of the fluid flow path through the housing 301. The air then passes through the filter element 376 (see FIG. 4) located in the base 314, where it then passes through a grate 388 in the bottom of the lower outer housing 311 (see FIG. 4). The air then passes through an opening 602 in the lower inner housing 511 and is drawn into the noise attenuation chamber 620 through the opening 602 (the directional arrows in FIG. 11 indicate the airflow path of fluid moving through the illustrated portion of the lower inner housing 511).

The noise attenuation chamber 620 is situated on the top portion of the deck 504 on the lower inner housing 511. In this embodiment, the noise attenuation chamber 620 is shown in the form of an expansion chamber. Accordingly, the air expands in the noise attenuation chamber 620 before being drawn into the blower 610 through an opening 605. The noise attenuation chamber 620 may abate or lessen the amount of noise produced by the positive airway pressure therapy apparatus 310.

The chamber 620 may be made of any material. In some embodiments, one or more walls defining the chamber 620 (as well as the chamber 710 described below) are formed from a material having properties for absorbing sound such as RTP 199X106571 Polypropylene High Gravity Compound available from RIP Company of Winona, Minn. USA. Other components of the inner housing 501 and outer housing 301 may also be selected (e.g., based upon density or other material properties) to abate or absorb noise and/or vibration.

Other types of noise attenuation chambers that utilize sound wave reflection and sound cancelling properties other than an expansion chamber (e.g., a muffler, resonator such as Helmholtz resonator, or multiple components tuned for broadband frequencies or multiple discrete frequency ranges) can also be used. In some embodiments, the density and/or other material properties used for the inner housing 501 are selected based on the noise abatement properties of the material. In yet other embodiments, the density and/or other material properties used for the material in the noise attenuation chamber 620 are varied with respect to the density and/or other material properties of the material associated with the upper outer housing 312 (shown in FIG. 4), the lower outer housing 311, or the base 314.

As shown in FIG. 4, attached to the bottom of the deck 504 of the lower inner housing 511 is the printed circuit board 650. The printed circuit board 650 may include various electronic components and electronic devices beneficial to operation of the therapy apparatus 310. The electronics and specifically the printed circuit board 650 may be contained in a sealed compartment such that they are not in contact with air in the airflow path. The boots 660, 665 may assist in isolating the circuit board 650 and other portions of the apparatus 310 from the airflow path. For example, the boots may surround portions of the inner housing 501 and define at least part of the airflow path from the filter element 376 to the blower outlet 360.

FIG. 12 is a rear or back view of the nose cone 711 defining yet another, e.g., second, noise attenuation chamber 710 associated with the housing 301 of the positive airway pressure therapy apparatus 310 according to an exemplary embodiment of the invention, while FIG. 13 is a side view. Both of these views illustrate the nose cone 711 detached from the outer housing 301 of the device 300. As illustrated in these views, the output of the blower 610 (see FIG. 11) is routed to the noise attenuation chamber 710 via an inlet 712. The noise attenuation chamber 710 may be formed within the nose cone 711, or alternatively by the nose cone 711 when the latter is attached to the forward end of the outer housing 301 (see FIGS. 3 and 4). In any event, the noise attenuation chamber 710 may define the outlet 360 of the outer housing 301 and the blower 610. Stated alternatively, the noise attenuation chamber 710 may be located between the blower 610 and the air outlet 360. The outlet 360, in turn, attaches to the inlet (proximal hose connector 475) of the delivery tube 318 as shown in FIG. 8.

The noise attenuation chamber 710 may utilize any one of a number of sound wave reflection and cancelling techniques (e.g., an expansion chamber, muffler, resonator such as a Helmholtz resonator, or use multiple components tuned for broadband frequencies or multiple discrete frequency ranges). As with the chamber 620, the noise attenuation chamber 710 may also use sound absorption techniques through use of materials that absorb sound energy such as plastics having special density and/or other material properties. As shown in FIG. 12, the inlet 712 to the noise attenuation chamber 710 is centered as is the outlet 360 as shown in FIG. 13. In other embodiments, the inlet 712 to, or the outlet 360 from, the noise attenuation chamber 710 may be off-center. In still further exemplary embodiments, both the inlet 712 and outlet 360 from the noise attenuation chamber 710 may be off-center and aligned, or off-center and misaligned, with one another. Of course, the alignment or misalignment of the inlet 712 and the outlet 360 of the noise attenuation chamber 710 may be selected to attenuate sound, and in some embodiments, may be selected to accomplish the most desirable noise attenuation.

In the embodiment shown in FIGS. 3-12, the upper outer housing 312 includes two expansion chambers 620 and 710, both of which abate noise produced by the flow of fluid along the airflow path. In some exemplary embodiments, the noise attenuation chambers may additionally utilize any one of a number of sound wave reflection and cancelling techniques such as an expansion chamber, muffler, resonator such as a Helmholtz resonator, or use of multiple components tuned for broadband frequencies or multiple discrete frequency ranges to further suppress or abate sound or noise emanating from the apparatus 310.

In other embodiments, the noise attenuation chamber 710 may include filter media 709 (see FIG. 13) in or near the chamber 710 that adds additional filtering properties for removing particles from the air as an addition to filter element 376. The filter media 709 (as well as the media used for element 376) may provide bacterial-static properties that may remove bacteria and viruses from the air delivered to the user. In addition, the filter media may also be selected to provide noise attenuation characteristics and/or abate noise. The nipples 725 and 730 may form a pressure sensing passage extending through the expansion chamber 710 and the filter media 709. The pressure sensing passage may be fluidly sealed from the expansion chamber 710 so that pressure/flow within the chamber is isolated from pressure/flow within the sensing passage.

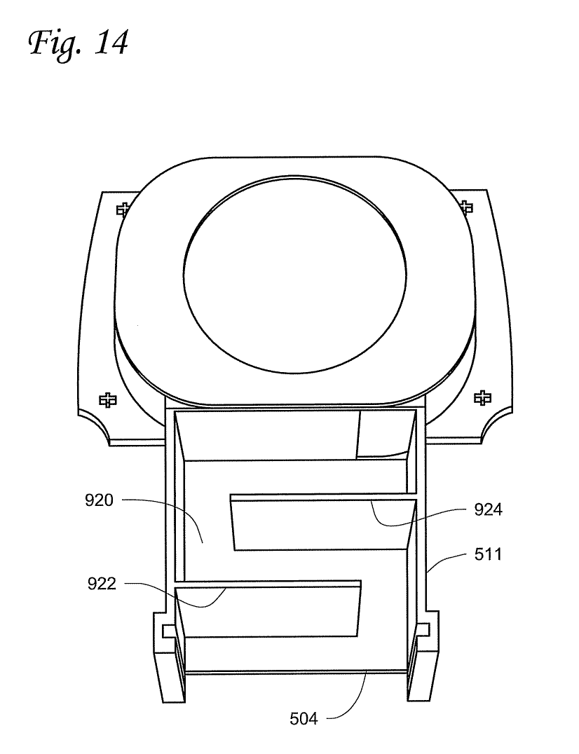

FIG. 14 is a perspective view of alternative embodiment of a noise attenuation chamber 920 that is a combination of an expansion chamber and a noise absorbent design. The noise attenuation chamber 920 is similar in some respects to the attenuation chamber 620 shown in FIG. 11 and, in fact, may be incorporated into or attached to the deck 504 of the lower inner housing 511. However, unlike the noise attenuation chamber 620, the noise attenuation chamber 920 may also include at least one baffle or wall 922, e.g., a plurality of walls 922 and 924. The walls 922, 924 may be placed in such a manner as to force the fluid in the airflow path to take a tortuous path. The tortuous path through the chamber 920 may provide additional noise abatement when compared to the expansion chamber 620 shown in FIG. 11.

Of course, the material used in the walls and sides of the noise attenuation chamber 920 can provide a density and/or other material properties that also abate noise, e.g., plastics, foams, or other similar materials. The walls 922, 924 can also be selected to have a different density or possess other material properties than the base, inner, and/or outer housings of the positive airway pressure therapy apparatus. In addition, it should be noted that the noise attenuation chamber 920 can have more than two walls. In fact, the noise attenuation chamber 920 could have any number of walls without departing from the scope of the invention. Ideally, the plurality of walls may be placed in a manner that results in desirable noise attenuation. For example, the plurality of walls 922, 924 may alternate to further attenuate noise.

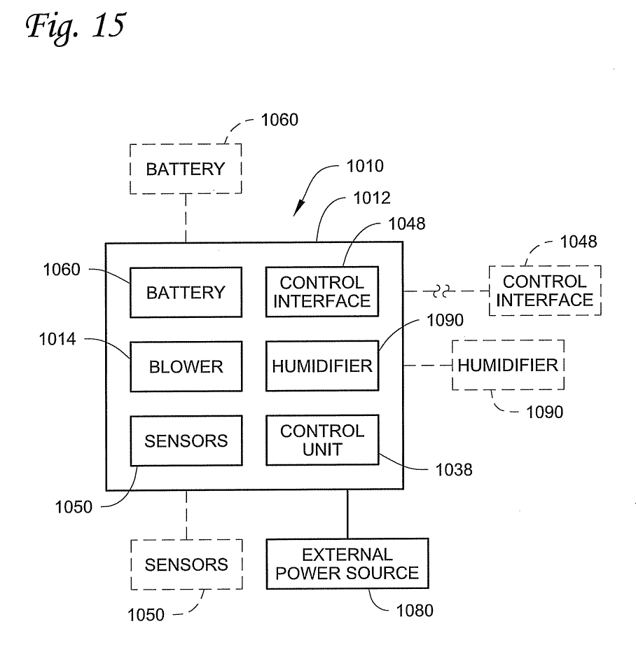

FIG. 15 is a schematic diagram of a positive airway pressure therapy apparatus 1010, which represents a part of any one of the apparatus 10, 110, 310 already described herein. To simplify the description, however, the apparatus 1010 will be correlated, where beneficial, with the apparatus 310 (e.g., with the device 300) as illustrated in FIGS. 3, 4, and 10-13. This is not to be considered limiting in any way, as the schematic depicted in FIG. 15 is also applicable to any other apparatus in accordance with embodiments of the present invention, including but not limited to the apparatus 10 of FIG. 1 or the apparatus 110 of FIG. 2.

As illustrated in FIG. 15, the various components of the apparatus 1010 may be positioned in, or remote from, the housing (e.g., outer housing 301) in accordance with various embodiments of the present inventions. A blower 1014, control unit 1038, control interface 1048, battery 1060, humidifier 1090, and various sensors 1050 may be secured to housing 1012. Alternatively, various components, e.g., the control interface 1048, battery 1060, humidifier 1090, and sensors 1050 (e.g., a humidity sensor located in the delivery tube 318 or mask 316) or components thereof may be remote from the housing 1012 as represented in broken lines in FIG. 15.

In one exemplary embodiment, the housing 1012 includes a sensor or switch 1050 (see also FIG. 4) associated with the housing (e.g., mounted to the base 314) and which serves to determine if the housing (e.g., outer housing 301 of the device 300) is position on the user's body, e.g., on the head. The switch could be a small micro-switch that is positioned so that it engages when the positive airway pressure apparatus 1010 (e.g., the device 300 of the apparatus 310) is on the user's head, and disengages when the apparatus is removed from the head. In the alternative, a sensor may be used. The sensor could be a photo sensor or an infrared sensor positioned so as to detect heat levels associated with a human body. It is contemplated that one more of such mechanisms could be used to determine if the positive airway pressure apparatus 1010 (e.g., 310) is removed from the user's body.

The sensor or switch may be useful to, for example, indicate therapy conclusion and automatically trigger a dehumidification or drying mode of the apparatus 1010. For example, therapy conclusion may be detected when a portion (e.g., the housing 1012 containing the blower 1014) of the positive airway pressure apparatus 1010 is removed from the user's head or body as detected by the sensor or switch 1050. Alternatively, therapy conclusion could be determined by detecting extended cessation of one or both of inhalation and exhalation using, for example, a flow or pressure sensor, or a parameter (e.g., speed, current draw) of the blower motor. Regardless of the mechanism used to detect therapy conclusion, the control unit 1038 could be configured to activate the drying mode. In the drying mode, power to the blower 1014 may continue, preferably at a reduced blower speed.

To reduce blower speed, the blower motor may be changed from a pressure control mode that is used during therapy (wherein motor/blower speed is controlled based upon feedback from a pressure sensor located in the mask or elsewhere), to a constant low speed (or, alternatively, a constant low air flow) mode. In other embodiments, reduced blower speed could be accommodated by delivering a constant lower voltage (or constant lower current) to the blower motor. By maintaining a reduced motor speed during the drying mode, unintended acceleration of the blower and motor (and/or accompanying high power consumption, noise, and vibration) may be avoided, while ensuring that a flow of air (via the outlet of the blower) is passed through the delivery tube 318 and mask 316 at a rate adequate to dehumidify or dry components of the apparatus 1010 (e.g., the delivery tube and mask).

Power to the blower 1014 could then be terminated upon completion of the drying mode. For example, the control unit 1038 could command the blower 1014 (e.g., a motor of the blower 610) to stay in such a drying mode for a period of time, after which blower power is automatically terminated. In some embodiments, the period of time may be a predetermined time interval. In another embodiment, the period of time could vary. The positive airway pressure apparatus 1010 could also be provided with another sensor that could detect moisture to determine when the device is adequately dry. For example, there could be included a humidity sensor in the delivery tube 318 or mask 316 that sends a command that is used to terminate the drying mode of the apparatus 1010 (e.g., 310) upon determining when at least one of the delivery tube and mask have reached a predetermined humidity level using one or more humidity sensors. It is further contemplated that a sensor could be provided that determines a lack of breathing (e.g., lack of inhalation and exhalation) and uses such detection to enable initiation of the drying mode.

The control unit 1038 may be a dedicated controller or a microprocessor that includes a processing portion and associated memory. The control unit 1038 may be capable of executing an instruction or set of instructions in the form of software or firmware.

A power source 1080 is typically required to power the components of the positive airway pressure apparatus 1010 (e.g., 310). The power source 1080 may be a household electrical outlet providing alternating (AC) current or may be the battery 1060. The power source 1080 may be external or remotely locatable, or may be secured to the housing 1012 (e.g., 301). In certain aspects, a direct current (DC) converter may be provided to appropriately convert an alternating current to a direct current which is typically utilized by the components of the positive airway pressure therapy apparatus 1010 (e.g., 310). The DC converter may be positioned at any convenient location on the power supply cord to permit its positioning on an adjacent bedside table, under the pillow of the user, on the floor, or elsewhere.

When the power source 1080 includes a battery 1060, the battery 1060 may be directly connected to the DC converter while the converter is connected to a household electrical outlet and function as a backup power source in the event of a power outage. Alternatively, the battery 1060 may be configured to be the sole source of power for the components of the positive airway pressure apparatus 1010. When connected to a household electrical outlet, the battery 1060 may be maintained in a fully charged condition until a power outage. If the external power source is not provided or fails, the battery 1060 may power the blower 1014 (610) and other components until power from the primary external power source 1080 is regained or the battery 1060 has discharged.

When securable to the housing 1012 (e.g., 301), the battery 1060 may be typically removable from the housing for purposes of recharging or replacement. In one aspect, the battery 1060 may include at least one interlock to removably secure the battery 1060 within the housing 1012. In other aspects, the battery 1060 may be remotely located and electrically connected to the components of the positive airway pressure apparatus 1010 by a cable. In this configuration, electrical connectors are typically provided on the cable and the positive airway pressure therapy apparatus 1010 to communicate electricity to the various components of the positive airway pressure therapy apparatus.

The humidifier 1090 may be provided to communicate moisture and/or other therapeutic agents into the pressurized air passing through the air delivery passage to humidify the pressurized air delivered to the user. The moisture provided by the humidifier 1090 may be in the liquid phase or the vapor phase, e.g., may be in the form of water vapor, liquid water droplets, mist, micro-droplets, fog, or various combinations of liquid water and water vapor. The pressurized air may be humidified for therapy, comfort, or other reasons, as will be recognized by those skilled in the art. The humidifier 1090 may be secured to or within the housing 1012 (e.g., 301), or may be located remotely. The humidifier 1090 generally includes at least a humidifier reservoir and, in certain configurations, a humidifier pump or humidifier heater. Various humidifiers and therapy apparatus incorporating the same are described in more detail in U.S. Pat. App. Pub. No. US 2007-0277825-A1.

In an alternative embodiment, the humidifying function may take the faun of a heat and moisture exchange (HME) element 390 (shown in FIGS. 3 and 8) positioned in the air delivery passage 28, e.g., located near the mask shell 410 and or near a distal end of the delivery tube 318. The material properties of the RIME element 390 may be such that it absorbs heat and moisture from exhaled air during exhalation, much as the nose does during breathing. On the next inhalation, the heat and moisture retained by the HME element 390 are mostly transferred to the inhaled air. Consequently, heat and moisture are mostly preserved during breathing and active heated humidifiers may not be necessary. HME elements and therapy apparatus incorporating the same are described in more detail in PCT International Publ. No. WO2010/096467.

The control unit 1038 can be provided to control one or more components of the positive airway pressure therapy apparatus 1010. The control unit 1038 may be particularly adapted to control the blower 1014. The control unit 1038 may include one or more circuits and/or may include one or more microprocessors as well as a computer readable memory. The control unit 1038 may be positioned within or on the housing 1012 or may be otherwise positioned or located, including remotely. The control unit 1038 is typically configured to output one or more control signals to various components of the positive airway pressure apparatus 1010. The control unit 1038, in some aspects may be adapted to receive one or more signals from one or more components of the positive airway pressure therapy apparatus 1010. The control unit 1038 may process or otherwise utilize the signals from the components of the positive airway pressure therapy apparatus 1010 (e.g., 310) in formulating the one or more control signals output to various components.

As an example, the control unit 1038 may process information from a pressure transducer such as transducer 655 shown in FIG. 4 and control the blower 1014 (e.g., blower 610) in such a way as to maintain desired pressure during breathing.

In one aspect, the control unit 1038 may control the blower 1014 (e.g., 610) in response to information including commands from the control interface 1048. The control interface 1048 includes one or more input devices, such as buttons (see, e.g., switch panel 309 of FIG. 4) or a touch-screen for example, to enter inputs for controlling features of the apparatus 1010. The control interface 1048 may also include a display and/or indicator lights to convey information about the operation of the apparatus 1010 to the user or a health care professional. The control interface 1048 may be in communication (wired and/or wireless) with the control unit 1038 to transfer information to and/or from the control unit 1038.

The control interface 1048 may be secured, permanently or removably, to the housing 1012 or may be otherwise positioned on components of the apparatus 1010. The control interface 1048 may also be configured as a remote control, either wired or wireless, as will be recognized by those skilled in the art upon review of the present disclosure. When configured as remote control, the control interface 1048 typically includes a transmitter to transmit signals to a receiver associated with the control unit 1038 and may include a receiver to receive signals from the control unit 1038. The transmission may be RF, infrared, or other means. The control interface 1048 may include one or more buttons, switches, touch screens, or other controls for controlling the blower 1014 and associated components. Various control aspects and therapy apparatus incorporating the same are described in more detail in U.S. Pat. No. 8,020,557.

The positive airway pressure therapy apparatus 1010 (e.g., 310) may be configured to position a mask (e.g., 316) in communication with the airway of a user to provide pressurized air and/or other gases for positive airway pressure respiratory therapy. The therapy may provide constant or variable positive airway pressure to the airway of the user. The apparatus allows for increased mobility and allows the user to more freely change head and body positions during sleep and during the administration of a positive airway pressure therapy. The apparatus and methods discussed herein mitigate or substantially reduce the noise propagated by the delivery tube, and the blower. The apparatus may also provide a reduced length of hose, as compared to other therapy apparatus, for delivery of the positive airway pressure to the mask, and allows for a reduced size positive airway pressure therapy apparatus. The resulting device is thus more portable and may be less cumbersome to use than conventional therapy apparatus.

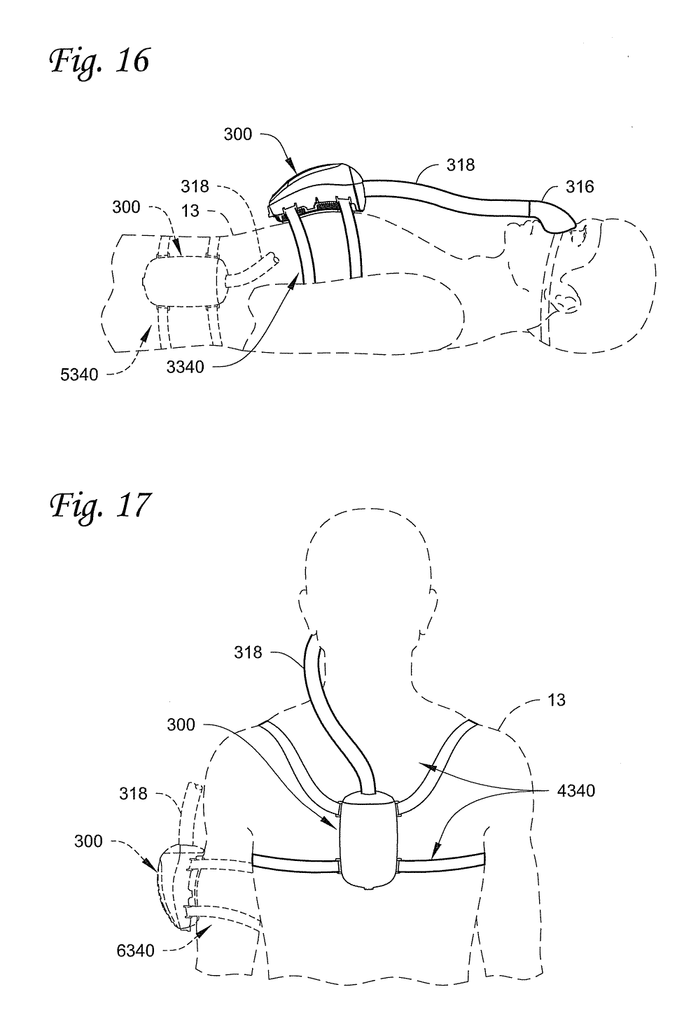

While described herein primarily in the context of a head-mounted blower apparatus, this is not limiting as embodiments of the present invention may find application to other body mounting locations as well as to remotely-(off the body) located blowers. For example, FIG. 16 illustrates the blower (e.g., device 300 of FIG. 3) attached with a strapping system 3340 to a chest area of a supine (or upright ambulatory) user 13 or, alternatively (see broken line rendering), to a waist of the user with a strapping system 5340. The apparatus (chest- or waist-mounted) may also include the delivery tube 318 and mask 316 as already described herein (other masks, e.g., nasal oxygen masks, as already known in the art may be substituted for the mask 316) to deliver pressurized air to the airway of the user. A simple strap, if needed, may secure the mask to the user's head.

FIG. 17 illustrates another body-mounted blower apparatus wherein the blower housing (e.g., device 300) is attached as a backpack module to be worn by an ambulatory user 13. The apparatus may again include a delivery tube 318 and a mask (not visible) to deliver pressurized air to the airway of the user. A strapping system 4340 may be used to secure the blower to the user's back, e.g., either directly as shown, or via a backpack enclosure (not shown). Alternatively, the device 300 could attach to the user's arm as shown in broken lines with a strapping system 6340. Such a system would still include the delivery tube 318 and mask in accordance with embodiments already described herein (or otherwise described in the art).

FIG. 18 depicts an illustrative portion of the wall 723 of the lower inner housing 511 (wall shown separated from the remainder of the housing 511) as already described herein with respect to FIG. 4. Similarly, FIG. 19 illustrates an exemplary end of the tube 490 incorporating the passive valve 492 as already described herein with respect to FIG. 8.

Illustrative embodiments are discussed and reference has been made to possible variations within the scope of this invention. These and other variations, combinations, and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. A positive airway pressure therapy apparatus comprising:
a device comprising a housing enclosing a blower, the housing configured to attach to a user's body, wherein the housing comprises at least two distinct side surfaces, and wherein each side surface defines an air inlet fluidly coupled to the blower;
a mask configured to communicate pressurized air generated by the blower to an airway of the user; and
a delivery tube extending from the housing to the mask, the delivery tube defining an air delivery passage configured to communicate the pressurized air from an outlet of the housing to an inlet of the mask.

2. The apparatus according to claim 1, wherein the housing is configured to attach to the body at a body location selected from the group consisting of the head, chest, back, arm, and waist.

3. The apparatus according to claim 1, wherein the blower is configured to deliver the pressurized air when the air inlet on a first side surface of the two distinct side surfaces is fully obstructed and the air inlet on a second side surface of the two distinct side surfaces is partially obstructed.

4. The apparatus according to claim 1, wherein the two distinct side surfaces comprise two parallel surfaces of the housing, each parallel surface comprising an elongate grate that forms the respective air inlet.

5. The apparatus according to claim 4, further comprising a third side surface connecting the two parallel surfaces of the housing, the third side surface also comprising an elongate grate forming an air inlet.

6. The apparatus according to claim 1, wherein the housing is configured to attach to a head of the user, and wherein the housing comprises a lower surface curved to conform substantially in shape to a shape of a surface of the head to which the housing is configured to attach.

7. The apparatus according to claim 1, wherein the housing is configured to attach to a head of the user, and wherein the housing comprises a pad having a shape that corresponds, or is deformable to correspond, to a shape of a surface of the head to which the housing is configured to attach.

8. The apparatus according to claim 7, wherein the pad comprises a biocompatible visco-elastic foam material.

9. The apparatus according to claim 7, wherein the pad comprises a friction-enhancing coating.

10. The apparatus according to claim 1, further comprising a filter element located within the housing proximate the air inlets.

11. The apparatus according to claim 1, further comprising a strapping system configured to secure the housing relative to the user's body.

12. A positive airway pressure therapy apparatus comprising:
a device comprising a housing enclosing a blower, the housing configured to attach to a user's body, wherein the housing comprises at least two distinct side surfaces, and wherein each side surface defines an air inlet fluidly coupled to the blower;
a mask configured to communicate pressurized air generated by the blower to an airway of the user;
a delivery tube extending from the housing to the mask, the delivery tube defining an air delivery passage configured to communicate the pressurized air from an outlet of the housing to an inlet of the mask; and
a strapping system configured to secure the housing to the body.

13. The apparatus according to claim 12, wherein the housing is configured to attach to the body at a body location selected from the group consisting of the head, chest, back, arm, and waist.

14. The apparatus according to claim 12, wherein the blower is configured to deliver the pressurized air when the air inlet on a first side surface of the two distinct side surfaces is fully obstructed and the air inlet on a second side surface of the two distinct side surfaces is partially obstructed.

15. The apparatus according to claim 12, wherein the housing comprises an outer housing and an inner housing contained within the outer housing, wherein the blower is at least partially supported relative to the inner housing by one or more resilient members.

16. The apparatus according to claim 12, wherein the at least two distinct side surfaces comprise three side surfaces.

17. A system for providing positive airway pressure therapy to a user, the system comprising:
a first mask seal configured to seal to an airway of the user;
a shell defining at least part of an air delivery passage configured to deliver pressurized air to the user; and
a first adapter positionable between the first mask seal and the shell, the first adapter configured to permit fluid tight attachment of the shell with the first mask seal.

18. The system according to claim 17, further comprising a second adapter of a configuration different than the first adapter, the second adapter configured to permit attachment of a second mask seal to the shell, wherein the second mask seal is of a configuration different than the first mask seal.

19. The system according to claim 18, wherein the first adapter and the second adapter each comprise a common universal end configured to attach to the shell.

20. The system according to claim 18, wherein the first adapter and the second adapter comprise a first end configured to receive the first mask seal and the second mask seal, respectively.
